(19) United States
(12) Patent Application Publication
(10) Pub. No.: US 2023/0001117 A1
(43) Pub. Date: Jan. 5, 2023

(54) DEVICE, PROCESS AND COMPUTER PROGRAM FOR DETERMINING A PATIENT COMPONENT

(71) Applicant: Drägerwerk AG & Co. KGaA, Lübeck (DE)

(72) Inventor: Marcus EGER, Lübeck (DE)

(21) Appl. No.: 17/843,403

(22) Filed: Jun. 17, 2022

(30) Foreign Application Priority Data
Jun. 18, 2021 (DE) 10 2021 115 865.6

(51) Int. Cl. A61M 16/00; A61B 5/00

(52) U.S. Cl. CPC A61M 16/0003; A61B 5/725; A61M 2016/0015; A61M 2016/0027

(57) ABSTRACT

A device for determining a patient component of an exchange of gas of a patient being ventilated, a measuring device or ventilation device with such a device for determining a patient component of an exchange of gas of a patient being ventilated, a process as well as to a computer program determine a patient component of an airway flow of a ventilation gas of a patient being ventilated. A determination of a patient component of an airway flow of a ventilation gas of a patient being ventilated are described. The determination includes an implementation with a signal processor, of forms a high-pass characteristic.

### CROSS REFERENCE TO RELATED APPLICATIONS

This application claims the benefit of priority under 35 U.S.C. § 119 of German Application 10 2021 115 865.6, filed Jun. 18, 2021, the entire contents of which are incorporated herein by reference.

### TECHNICAL HELD

The present invention pertains to a device, to a process as well as to a computer program for determining a patient component of an airway flow of a ventilation gas of a patient being ventilated, especially but not exclusively to a concept for determining the patient. component of an airway flow on the basis of the time coarse (time course) of a respiratory muscle pressure. The present invention pertains, moreover, to a ventilation device, as well as to a measuring device for determining the patient component of the airway flow.

### TECHNICAL BACKGROUND

The maintenance and recovery of spontaneous breathing had a high priority for a long time in intensive care. In case spontaneous breathing, is not possible for the patient, a lung-protective ventilation is employed, which shall damage the pulmonary tissue as little as possible. The protection of the respiratory muscles, especially of the diaphragm, has only recently moved into the focus.

Details concerning the background and the state of the art of the present invention are described, for example, in the following documents (which are hereby incorporated by reference): U.S. Pat. Nos. 5,820,560 B1, 7,021,310 B1, WO 2019 154 834 A1, WO 2019 154 837 A1, WO 2019 154 839 A1, WO 2020 079 266 A1, WO 2020 188 069 A1, DE 10 2019 006 480 A1, US 2017 0252558 A1, US 2015 0366480 A1, US 2012 0103334 A1, DE 10 2019 007 717 B3, US2021205561 (A1), DE 10 2007 062 214 B3, WO 2018 143 844 A1, DE 10 2015 011 390 A1, US 2022 072 249 (A1), US 2009 114 224 (A1).

Reference shall, furthermore, be made to the publications listed below (which are hereby incorporated by reference) in connection with the background of the present invention concerning the determination of a patient component of an airway flow of a ventilation gas of a patient being ventilated. The sources mentioned below provide additional information on the technical, medical engineering, medical as well as clinical background. The following list comprises—in an incomplete form—an exemplary selection of documents and publications on the detection and processing of different measured signals or signals in/at the human body and the use thereof within the framework of a ventilation, a ventilation control as well as in the field of ventilation with breathing stimulation.
Walker, D. J. “Prethction of the Esophageal Pressure by the Mouth Closing Pressure Following Magnetic Stimulation of the Phrenic ,” Dissertation, University of Freiburg. 2006.
Kahl, L, et al. “Comparison of Algorithms to Quantify Muscle Fatigue in Upper Limb Muscles Based on sEMG Signals,” Medical Engineering Physics, 2016.
Jansen, D. et al.: “Estimation of the Diaphragm Neuromuscular Efficiency Index in Mechanically Ventilated Critically III Patients,” Critical Care, 2018.

Liu, L. et al.; “Neuroventilatory Efficiency and Extubation Readiness in Critically III Patients,” Critical Care, 2012.
Cattapan, S. E. et al.: “Can Diaphragmati. Contractility Be Assessed by Airway Twitch Pressure in Mechanically Ventilated Patients?,” Thorax, 2003.
Younes, M. et al,: A Method for Monitoring and Improving Patient Ventilator Interaction,” Intensive Care Med. 2007.
Blanch Lluis et at.: “Measurement of Air Trapping. Intrinsic Positive End-Expiratory Pressure and Dynamic Hyperinflation in Mechanically Ventilated Patients,” Respiratory Care, 2005.
Purro, A. et al.: “Static Intrinsic PEEP in COPD Patients During Spontaneous Breathing,” AJRCCM, 1988.
Bernardi, E. et al.: “A New Ultrasound Method for Estimating Dynamic Intrinsic Positive Airway Pressure; A Prospective Clinical Trial,” AJRCCM, 2018.
Pisani, L. et al.: “Noninvasive Detection of Positive End-Expiratory Pressure in COPD Patients Recovering, from Acute Respiratory Failure,” European Respiratory Journal, 2016.
Bellani, G. et al.: “Clinical Assessment of Auto-positive End-Expiratory Pressure by Diaphragmatic Electrical Activity During Pressure Support and Neurally Adjusted Ventilatory Assist,” Anesthesiology,” 2014.
Younes, M.: “Dynamic Intrinsic PEEP (PEEPi,dyn): Is It Worth Saving?” AJRCCM, 2000.
Shalaby, R. E.-S.: “Development of an Electromyography Detection System for the Control of Functional Electrical Stimulation in Neurological Rehabilitation,” Dissertation, 2011.
BELLANI, G. ET AL.:”“The Ratio of Inspiratory Pressure Over Electrical Activity of the Diaphragm Remains Stable During ICU Stay and is Not Related to Clinical Outcome” RESPIRATORY CARE; APRIL 2016: VOL 61 NO 4.
Putensen, C. et al.: “Long-Term Effects of Spontaneous Breathing During Ventilatory Support in Patients with Acute Lung Injury” AM Journal Respir. Crit. Care Med.,” 2001.
Otis, A. B. et al.: “Mechanics of Breathing in Man,” J. Appl. Physiol., 1950.
Osuagwu, B. A. C. et al.: “Active Proportional Electromyogram Controlled Functional Electrical Stimulation System, Nature, 2020.
Virtanen, J. et al.: “Instrumentation for the Measurement of Electric Brain Responses to Transcranial Magnetic Stimulation,” Med. Biol. Eng. Comput., 1999.

Reference will be made at times to these and other documents in connection with special aspects of the present invention in the course of the description.

To avoid ,a lack of clarity based on linguistic wordings, some references should be made at the beginning of the application for the understanding as well as some explanations shall be given concerning the use of terms. In the description and/or in the patent claims, the wordings in the verbal form, nominalized verbal form, which are used in the process and also due to embodiments of the control unit in embodiments of the devices, which embodiments are according to the present invention, for example and especially “a determination,” “a high-pass filtering,” “a determining,” “a stimulating,” “a performing,” “a transforming,” “an outputting,” “a detecting,” are used within the framework of the present invention or inventions with the same meaning as wordings with nouns in the nominalized form,. for example and especially “a determination,” “a high-pass filtration,” “a determination,” “a performance,” “an output,” “a detection,” “a setting,” “a stimulation.” Equivalent and similarly acting meanings are obtained in respect to the disclosure of the invention or inventions for the wordings with nouns, nouns, nominalized verbs and verbs, so that reference is or can also always mutually be made for features and details between the verbal form and the nominalized form concerning the disclosure. Wordings with “performances of determinations, high-pass filtrations, stimulations, stimulatings, determinings, detectings, inputs, outputs, transformations, etc.” shall also be considered to be included in the equivalent and similarly acting meanings. Features and details that are described within the framework of the present inventions in connection with devices and embodiments of devices also apply, of course, in connection with the processes within the framework of the present invention and of they embodiments thereof as well as vice versa, so that reference is and can always mutually be made to the individual aspects of the present invention concerning the disclosure.

For example, DE 102019006480 describes a process which makes possible an estimation of components of the work of breathing by means of electromyography of the respiratory muscles.

Electromyography (EMG) is a neurological examination for living beings, by which the natural electrical activity of a muscle is measured. Electromyography (EMG) makes it possible to determine the force with which a muscle is tensioned. Measurements on superficial muscles are also called sEMG. Electrical impedance myography (EIM) is a non-invasive technique for assessing the health of muscles, whereby the properties of individual muscles or muscle groups or even the composition of muscles and microscopic structures of muscles can be investigated. Myomechanography (MMG) is a method for detecting elastic, viscous and plastic qualities of muscles. The parameter Pmus represents a variable derived from an EMG signal (electromyogram), an sEMG signal (surface electromyogram), an EIM signal (electrical impedance myogram) or an MMG (mechnomyogram) signal, which signals are detected by measurement. The parameter Pmus indicates here a pressure level, which has been elicited on the basis of a muscle breathing effort of a patient. The cause of the muscle breathing effort may have been initiated by the patient himself in the form of a spontaneous breathing activity and/or it may have been elicited by means of an external, for example, electrical, magnetic or electromagnetic stimulation. The muscle breathing effort may be derived, on the one hand, indirectly from electrical, electromagnetic or magnetic signals. Muscle breathing efforts may also be detected directly by measurement as a pressure difference against a reference pressure, for example, by means of a pressure measurement oar the thorax of a patient. The ambient pressure or also a pressure level that is provided by a ventilator may be selected as a reference pressure. Pressure levels typically provided by ventilators are, for example, an inspiratory pressure level, usually called inspiratory pressure or inspiration pressure Pinsp, as well as, for example, an expiratory pressure level, usually called expiratory pressure or expiration pressure Pexp; a special case of an expiratory pressure level is represented by the so-called PEEP (positive end expiratory pressure), which describes a pressure level which can be detected by measurement at the end of the exhalation in the airways of the patient as a pressure difference against the ambient pressure. Both the inspiration pressure Pinsp and the expiration pressure Pexp are detected as pressure differences against the ambient pressure and are usually stated in the unit mbar. Such a parameter Prints may also be called a respiratory muscle pressure Pmus.

The terms “muscle airway pressure,” “respiratory muscle pressure” are used in the description and/or in the patent claims within the framework of the present invention or inventions with wordings such as “parameter Pmus,” “pressure parameter Pmus,” “pressure parameter or parameter P, Pmus, which indicates a pressure which has been caused by a muscle breathing effort of a patient,” “a breathing pressure Pmus generated by the muscles of a patient” as terms haying the same meaning and producing the identical effect, so that reference can mutually be made concerning the term.

The parameter Flowmus represents a variable derived from the parameter Pmus. Higher frequencies in the signal curve of the parameter Pmus may provide an indicator of muscle-related components, flow direction and reversal of the flow direction of the airway flow. The high-pass filtering of the parameter Pmus may be used to determine the parameter Flowmus. Flow rates that flow into the patient, are inhaled, or which flow out of the patient during phases of exhalation, i.e., are exhaled, are designated by the term airway flow. The parameter Flowmus indicates here a flow rate with a flow direction, wherein the cause of the flow is based on a muscle breathing effort of a patient. The cause of the muscle breathing effort may have been initiated by the patient himself in the form of a spontaneous breathing activity and/or it may have been elicited by means of an external, for example, electrical, magnetic or electromagnetic stimulation. Such a parameter Flowmus may also be called a muscle airway flow or even as a respiratory muscle flow Flowmus. The signal processing with high-pass signal filtering of the signal curve of the parameter Pmus makes it possible to determine/find muscle-related breathing phase changes and times in the parameter Flowmus, at which a reversal of the sign of the parameter Flowmus takes place. The reversal of the sign of the parameter Flowmus indicates here times at which a breathing phase change occurs between inhalation phases (inhalation) and exhalation phases (exhalation), which are based on muscle breathing efforts of the patient.

The terms “muscle airway flow,” “respiratory muscle flow” are indicated in the description and/or in the patent claims within that framework. of the present invention with wordings such as “parameter Flowmus,” “flow parameter Flowmus,” flow parameter or parameter flow, Flowmus, which indicates a pressure, which has been caused by a muscle breathing effort of a patient,” “a flow rate produced by a muscle breathing effort of a patient, Flowmus” as terms having the same meaning and producing the identical effect, so that reference can mutually be made concerning the term. The following list is used to clarify some of the terms used within the framework of this application:
An airway pressure is defined in the sense of the present invention as a pressure or as a pressure level—mostly and usually above the ambient pressure—in the airways, in the lungs, in the trachea of a living being.
A muscle airway pressure or a respiratory muscle pressure is defined in the sense of the present invention as a component of the airway pressure produced by muscles, especially on the basis of spontaneous or stimulated muscle activity of the respiratory muscles and/or muscle activity of the auxiliary respiratory muscles of the living being.
An airway flow or a breathing gas flow is defined in the sense of the present invention as any movement of quantities of breathing gas as a quantity of inhaled gases to and into a being as well as as a quantity of gases exhaled by a living being.
A muscle airway flow (or a respiratory muscle flow) is defined in the sense of the present invention as any movement of quantities of breathing gas as a quantity of inhaled gases to 1.0 and into a living being as well as as a quantity of gases exhaled from and by a living heirs on the basis of —spontaneous or stimulated—muscle activity of the respiratory muscles and/or muscle activity of the auxiliary respiratory muscles of the living being.

The terms muscle airway pressure and respiratory muscle pressure are used synonymously within the framework of the present invention.

The terms muscle airway flow and respiratory muscle flow are used synonymously within the framework of the present invention.

The respiratory muscles comprise the main muscles acting during inhalation, the diaphragm, and the auxiliary muscles. These include, among other things, the external intercostal muscles (acting during inhalation) and the internal intercostal muscles (acting during exhalation) and the abdominal muscles acting during exhalation. It was thus determined that the diaphragm becomes atrophied due to long ventilation times and an excessively high level of support of the spontaneous breathing, making a complicated weaning necessary. On the other hand, the respiratory muscles may become exhausted and damaged (fatigue) by increased respiratory load (obstruction, restriction). Some patients tend to have in certain situations excessively great intrinsic efforts, which may, in turn, damage the lungs.

It is described in the state of the art how respiratory muscles can be stimulated. All muscles can be stimulated directly by activation of the muscle fibers or of the supplying efferent nerves. For example, the muscle fibers of the diaphragm can be stimulated directly transcutaneously. As an alternative, the phrenic nerve, which is responsible for the contraction of the diaphragm, can be stimulated. Activation of the muscles and contraction occur in both cases. The goal of these methods is to improve the weaning, to promote the removal of secretion and possibly also to avoid ventilation or breathing assistance. Unlike in the case of ventilation or assisted spontaneous breathing, administration of a breathing gas is not necessary in this case.

A flow and pressure sensor is used in US 20170252558A1 as well as in Cattapan, S. E. et al,: “Can Diaphragmatic Contractility Be Assessed by Airway Twitch Pressure in Mechanically Ventilated Patients?” Thorax, 2003 to determine the work of breathing of the patient and to adapt the stimulation such that a time corridor is obtained. However, there is no information technological connection to a ventilator. Parameters of the mechanics of breathing must be taken from the graphic user interface of the ventilator and be entered manually into the separate stimulator. It is, furthermore, not possible to rely on the indicated values of the mechanics of breathing, which are calculated from pneumatic signals, in conventional ventilators as long as the patient is breathing spontaneously to a great degree, i.e., the determined indicator of the work of breathing is only a rough estimate.

Accordingly, there is no method so far that can adequately adjust and coordinate the ventilation and the stimulation especially in view to the work of breathing to be performed. It is self-evident and known, for example, from WO 2019154834 (which is hereby incorporated by reference). WO 2019154837 which is hereby incorporated by reference); WO 19154839 (which is hereby incorporated by reference), WO 2020079266 (which is hereby incorporated by reference), and US 2017/0252558A1, that stimulation and ventilation must be coordinated in their basic mechanism. However, there is no method so far that can predefine the degree of assistance and of stimulation, e.g., depending on a therapy, goal. The minute. volume necessary for the patient as well as threshold values and limits of the mechanical pressure assistance (triggers, pressures, frequencies) are usually set. The component to be contributed by the patient can hardly be set, because there is no sufficiently precise possibility so far for splitting the work of breathing between machine and patient. A process in which mechanics of breathing parameters of the ventilator used are transferred manually and are then used in the stimulator is practically unaffordable: The parameters change e.g., after repositioning of the patient. Consequently, they would have to be entered repeatedly. In addition, the are very inaccurate as long as only pneumatic signals were used in the ventilator for determining the parameters. Consequently, the actual contribution of the patient is hardly known, since it would be necessary for this to know the contribution of the patient to the driving pressure (Pmus) or breathing gas flow (FlowMus). DE 10 2019 006 480 describes the estimation of these work of breathing components by means of electromyography of the respiratory muscles.

The electrical activity of the diaphragm (EAdi) is recorded by means of a modified gastric probe equipped with electrodes in order to control the pressure assistance of the ventilator proportional y to this electrical activity in processes with “Neurally Adjusted Ventilatory Assist” (NAVA), as described, e.g., in: Sinderby et al.: “Is One Fixed Level of Assist Sufficient to Mechanically Ventilate Spontaneously Breathing Patients?,” Yearbook 0f Intensive Care and Emergency Medicine, 2007 (which is hereby incorporated by reference), as well as in Sinderby et at: “Neural Control of Mechanical Ventilation in Respiratory Failure,” Nature Medicine, 1999 (which is hereby incorporated by reference).

An especially proportionally assisting NAVA process with the use of a signal for the electrical activity of the diaphragm, whose peculiar feature is that the electrical activity of the diaphragm, which is needed for a defined tidal volume (the so-called neuroventilatory efficiency shall be maintained at a con-tan value by means of a “closed-loop”control, is known from U.S. Pat. No. 7,021,310 B1.

Variants of how, for example, the pressure parameter Pmus or the “muscle airway pressure”or respiratory muscle pressure Pmus, pₘᵤₛ(t) can be determined arise from US 2009 159 082 A1 (which is hereby incorporated by reference) and DE 10 2007 062 214 133, to the explanations of which reference shall also expressly made in the description of this application concerning the disclosure concerning terms such as “muscles,” “respiratory muscles,” “muscle airway pressure,” “respiratory muscle pressure,” “parameter Pmus,” “pressure parameter Punts,” “pressure parameter or parameter Pmus, which indicates a pressure that has been caused by a muscle breathing effort of a patient.”
a) Calculation from measured values for airway pressure, volume flow Flow(t), from which the tidal volume Vol(t) is also obtained by integration, as well as from the lung mechanical parameters R (resistance) and E (elastance).
b) Determination by equating with the negative airway pressure-Pₒₖₜ(t) measured during an occlusion, wherein the lung mechanical parameters R and E are either also calculated or are predefined.
c) Determination by means of an esophageal catheter, which is equipped with pressure sensors for measuring the intrathoracic pressure Pₑₛ(t). The esophageal catheter may optionally be configured and used for measuring the abdominal pressure P_{abd}(t).
d) Determination by means of an array of surface electrodes or sensors, which, arranged on the thorax, provide electromyographically mechanomyographicallv determined electrical signals, which can correspondingly also be related to the respiratory muscle pressure Pₘᵤₛ(t) by means of suitable assignment rules, tables, functions or conversion parameters, for example, the so-called neuromechanical efficiency, or “neuromuscular efficiency” (NME) and correspondingly also with a volume Vₘᵤₛ(t) by meals of the so-called “neuroventilatory efficiency” (NVE).
e) Determination by means of a gastric probe equipped with electrodes, which yield electrical signals, which can be related to the respiratory muscle pressure Pₘᵤₛ(t) by means of suitable assignment rules, tables, functions or conversion parameters.

The variants of the determination of Pₘᵤₛ(t) and Pmus listed in a) through e) and the additional components, such as sensors, electrodes, surface electrodes, pressure sensors, flow sensors, gastric probe, esophageal catheter, which are necessary for the determination of these variables in a device, in a system or in a process, are obtained corresponding to the variant. The breathing activity signal u_{eng}(t) can be subjected to a transformation into a pressure signal p_{eng}(u_{eng}(t)) by means of a predefined transformation rule. The transformation rule can be determined by linear or nonlinear regression between u_{eng}(t) and pₘᵤₛ(t) or also with other procedures, e.g., with neuronal networks, machine learning, or staple scaling. For example, the following linear regression equation can be used to determine the regression coefficients for the transformation rule being sought; pₘᵤₛ(t)=a₀+a₁*u_{engt}(t)+a₂*u²_{eng}(t) +a3*u³_{eng}(t)+(t), with which a transformed p_{eng}(t) signal is ultimately obtained for the further use, for example, for the ventilation control and/or for the stimulation.

### SUMMARY

Based on this, one object of the present invention is to provide an improved concept for determining a patient component of an airway flow of a ventilation gas of a patient being ventilated.

The object is accomplished by a device for determining a patient proportion of a gas exchange of a ventilated patient according to the invention, a measuring device or a ventilation with the device for determining a patient proportion of a gas exchange of a ventilated patient according to the invention, a process determining a patient component of a gas exchange of a patient being ventilated according to the invention and a computer program (computer readable media—software provided on a non transient tangible medium (or media)) with a program code for carrying out all or some of the process when the program code is executed on a computer, on a processor or on a program enable hardware component.

Advantageous embodiments of the present invention appear from this disclosure and will be explained in more detail in the following description partly with reference to the figures.

Exemplary embodiments are based on the discovery that a component of the airway flow (or of the volume=integral of the flow) of the total quantity of breathing gases inhaled by the patient within the airways, which is based directly on a spontaneous breathing activity of a patient, can be determined. The measured airway flow always also contains the component of the flow that originates from the assistance by the ventilator. It is a further discover that the respiratory muscle pressure Pmus, which is an indicator the breathing effort of the patient, describes the spontaneous breathing activity directly, because it represents the spontaneous component of the driving pressure.

Exemplary embodiments therefore create a process for determining a patient component of an airway flow of a ventilation gas of a patient being ventilated. The process comprises a determination of a time course of a respiratory muscle pressure of the patient and a high-pass filtering of the time course of the respiratory muscle pressure in order to obtain a time course of a filtered respiratory muscle pressure. The process comprises a determination of the patient component of the airway flow based on the time course of the filtered respiratory muscle pressure. Exemplary embodiments can thus determine a patient component of the airway flow based on the more rapid changes in the respiratory muscle pressure of a patient.

The process may comprise, for example, a transformation of the time course of the respiratory muscle pressure into a frequency domain and the high-pass filtering may take place in the frequency domain in order to obtain a filtered spectrum. Filtering in the frequency domain may be carried out by simply cutting out the spectrum. The process may then comprise a transformation of the filtered spectrum into a time domain in order to obtain the time course of the filtered respiratory muscle pressure. An efficient signal processing can thus be brought about via a frequency domain transformation.

The high-pass filtering may also be carried out in the time domain in some exemplary embodiments. This can make possible a more rapid filtering, because no block-by-block signal processing or signal processing of parts is necessary.

The patient component of the airway flow may comprise, e.g., a minute volume or a tidy volume, which is based on the spontaneous activity of the patient.

Different indicators, especially those preferred by the clinical staff, are conceivable in exemplary embodiments for stating. the patient component.

The determination. of the time course of the respiratory muscle pressure may be carried out, for example, oh the basis of an electromyographic signal, of a pneumatic signal or of a mechanical signal. For example, mechanomyographically detected signals, signals detected by means of a strain sensor and/or signals detected by means of an ultrasound sensor, may be used. Non-invasive signal detection methods may be advantageous here.

In further exemplary embodiments, the process may comprise a determination of a high-pass characteristic for the high-pass filtering from the time course of the respiratory muscle pressure. The filter characteristic can then advantageously be determined directly from the signal and thus be adapted to the signal curve. For example, the high-pass characteristic is determined by a time constant. This can then also be checked and adapted in the further course.

The determination of the high-pass characteristic can be carried out on the basis of a kinetic equation for the breathing circuit of the patient. Such a kinetic equation can describe a model of the breathing apparatus of the patient id thus be used as the basis for the determination of the filter characteristic. The process may comprise in some exemplary embodiments the carrying out of a linear regression, of a Kalman filtering or of an estimation method, An efficient means is thus available for the determination of the high-pass characteristic.

Determination of a time course of the patient component of the airway flow can be carried out in further exemplary embodiments. A change in the patients breathing component thus also becomes apparent and a trend diagram and further actions, for example, for adapting ventilation parameters, can be derived from this. A curve of FlowMus (with a high time resolution) can be used as the basis for further indicators, e.g., trigger event, minute volume, tidal volume, etc.

Moreover, determination of times at which a breathing effort of the patient begins from the patient component of the airway flow can be carried out in some exemplary embodiments. These times can then be used as triggers for further actions during the ventilation. Consequently, the process may also comprise an output of a trigger signal for a ventilator based on the times at which a breathing effort of the patient begins, Exemplary embodiments can thus achieve an improved synchronization between the spontaneous breathing activity of the patient and a ventilation assistance by a ventilator. In addition or as an alternative, a determination of times at which a breathing effort of the patient ends can analogously be carried out from be patient component of the airway flow. The process can then comprise an output of a cycling-off signal for a ventilator based on the times at which a breathing effort of the patient ends. These times may be used analogously for the synchronization or setting of ventilation parameters.

In further exemplary embodiments, the process may comprise a determination of a spontaneous respiration rate of the patient from the patient component of the airway flow. The spontaneous respiration rate may, for example, be outputted in order to be made available to the clinical staff or else to influence the further therapy or ventilation configuration.

A further exemplary embodiment is a computer program with a program code (computer-readable medium storing instructions) for carrying out one of the processes being described here when the program code is executed on a computer, on a processor or on a programmable hardware component.

Exemplary embodiments create, moreover, a device of a measuring device for a ventilation device and for determining a patient component of an airway flow and for determining a patient component of an airway flow of a ventilation gas of a patient being ventilated. The device comprises one or more interfaces, which are configured for the exchange of information with the measuring device or with the ventilation device. The device comprises a control unit, which is configured to determine a time course of a respiratory muscle pressure of the patient and for the high-pass filtering of, the time course of the respiratory muscle pressure in order to obtain a time course of a filtered respiratory muscle pressure. The control unit is configured to determine the patient component of the airway flow based on the time course of the filtered respiratory muscle pressure.

The control unit may be configured to carry out the determination of the time course of the respiratory muscle pressure on the basis of an electromyographic signal, of a pneumatic signal or of a mechanical signal. The device may comprise for this purpose electrodes for detecting the electromyographic signal, sensors for detecting a pneumatic signal or sensors for detecting a mechanical signal. For example, strain sensors or ultrasound sensors may be used for this purpose.

The control may be configured to carry out one or more of the process steps being described here.

Further exemplary embodiments are a ventilation device or a measuring device with a device being described here for determining a patient component of an airway flow of a ventilation gas of a patient being ventilated.

An embodiment according to the present invention shows a device for a measuring device or a ventilation device and for determining a patient component of a gas exchange of a patient being ventilated,
with one or more interfaces, which are configured for the exchange of information with the measuring device
or with the ventilation device,
with a control unit, which is configured
   to determine a time course of a respiratory muscle pressure of the patient,
   to carry out a high-pass filtering of the time course of the respiratory muscle pressure in order to obtain a time course of a filtered respiratory muscle pressure,
   and to determine the patient component based on the time course of the filtered respiratory muscle pressure.

In a preferred embodiment of the device, the control unit may be configured to carry out the determination of the time course of the respiratory muscle pressure on the basis of an electromyographic signal, of a pneumatic signal or of mechanical

In a preferred embodiment, the device may comprise
electrodes for detecting the electromyographic signal.
or sensors for detecting a pneumatic signal,
or sensors for detecting a mechanical signal.

In a preferred embodiment of the device, the control unit may be configured to carry out the high-pass filtering in the time domain in order to obtain the time course of the filtered respiratory muscle pressure.

In a preferred embodiment of the device, the control unit may be configured to carry out a transformation of the time course of the respiratory muscle pressure into a frequency domain.

In a preferred embodiment of the device, the control unit may be configured to carry out the high-pass filtering in the frequency domain in order to obtain a filtered spectrum.

In a preferred embodiment of the device, the control unit may be configured to carry out a transformation of the filtered spectrum into a time domain.

In a preferred embodiment of the device, the patient component of the gas exchange may comprise a minute volume or a tidal volume, which is based on the spontaneous activity of the patient.

In a preferred embodiment of the device, the control unit may be configured to determine a high-pass characteristic for an adapted performance of the high-pass filtering
on the basis of the time course of the respiratory muscle pressure
and/or on the basis of a kinetic equation for the breathing circuit of the patient.

In a preferred embodiment of the device, the control unit ay be configured to carry out a linear regression, a Kalman filtering, or an estimation method on the basis of the kinetic equation to determine the high-pass characteristic.

In a preferred embodiment of the device, the control unit may be configured to determine times at which a breathing effort of the patient begins from the patient component.

In a preferred embodiment of the device, the control unit may be configured to
to output a trigger signal for the ventilator and/or
to output a cycling-off signal for the ventilator

on the basis of the times at which a breathing effort of the patient begins,

In a preferred embodiment of the device, the control unit may be configured to determine a spontaneous respiration rate of the patient from the patient component.

A preferred embodiment may configure a ventilation device with a device based on the above-described embodiments.

A preferred embodiment may be formed by a process for determining a patient component of a gas exchange of a patient being ventilated, with the following steps:
Performance of a detection and determination of a time course of a respiratory muscle pressure of the patient on the basis of an electromyographic signal, of a pneumatic signal or of a mechanical signal,
performance of an adapted high-pass filtering to the time course of the respiratory muscle pressure in order to obtain a time course of a filtered respiratory muscle pressure, and
performance of a determination of the patient component of the airway flow based on the time course of the filtered respiratory muscle pressure:

In an especially preferred embodiment: a determination of a high-pass characteristic may he carried out for the adapted performance of the high-pass filtering
on the basis of the time course of the respiratory muscle pressure and/or
on the basis of a kinetic equation for the breathing circuit of the patient.

A preferred embodiment may be formed by a computer program (computer readable media—software provided on a non-transient, tangible medium (or media)) with a program code for carrying out the process when the program code is executed on a computer, on a processor- or on a programmable hardware component.

Some examples of devices and/or processes will be explained in more detail below with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTIO. OF THE DRAWINGS

In the drawings:

FIG. 1 is a flow diagram showing an exemplary embodiment of a process for determining a patient component of an airway flow of a ventilation gas of a patient being ventilated;

FIG. 2 is a schematic view of an exemplary embodiment of a device for a measuring device or a ventilation device and for determining a patient component of an airway flow of a ventilation gas of a patient being ventilated and of an exemplary embodiment of a measuring or ventilation device;

FIG. 3 is a graph showing a time course of a pressure generated by a ventilator, a volume flow between the ventilator and the patient, and a curve (course) of intrinsic breathing efforts of a patient (a patients own breathing efforts);

FIG. 4 is a graph showing examples of time courses (time courses) of ventilation signals, and

FIG. 5 is a graph showing further examples of time courses of ventilation signals.

### DESCRIPTION OF PREFERRED EMBODIMENTS

Different examples will be described now in more detail with reference to the attached figures. The thicknesses of lines, layers and/or areas may be exaggerated in the figures for illustration. Further examples may cover modifications, equivalences and alternatives, which fall within the framework of the disclosure. identical or similar reference numbers pertain in the entire description of the figures to identical or similar elements, which may be implemented in an identical or modified form, while they provide the same function or a similar function. It is obvious that if an element is described as being “connected” to or “coupled” with another element, the elements may be connected or coupled directly or via one or more intermediate elements. if two elements A and B are combined with the use of an “or,” this should be understood to be such that all possible combinations are disclosed, i.e., only A, only B or A and B, unless something else is explicitly or implicitly defined. An alternative wording for the same combinations is “at least one of A and B” or “A and/or B.” The same applies, mutatis mutandis, to combinations of more than two elements.

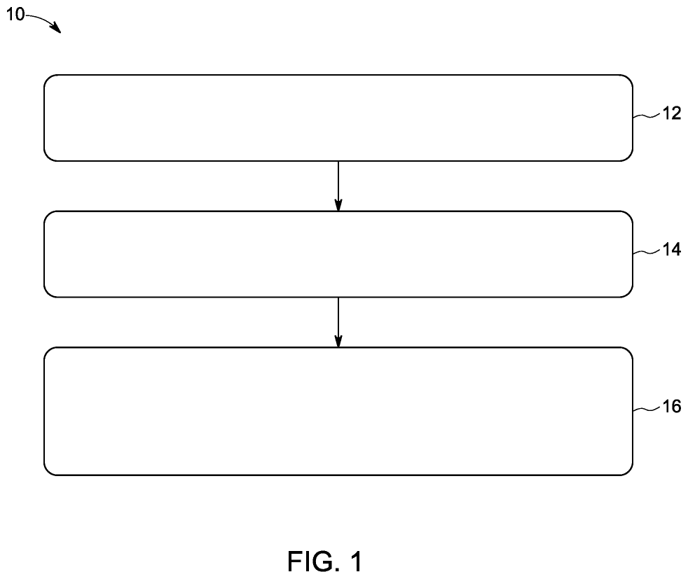

FIG. 1 shows an exemplary embodiment of a process 10 for determining a patient component of an airway flow of a ventilation gas of a patient being ventilated. The process 10 for determining the patient component of the airway now oldie ventilation gas comprises a determination 12 of a time course of a respiratory muscle pressure and a high-pass filtering 14 of the time course of the respiratory muscle pressure in order to obtain a time course of a filtered respiratory muscle pressure. The process 10 comprises a determination 16 of the patient component of the airway flow based on the time course of the filtered respiratory muscle pressure.

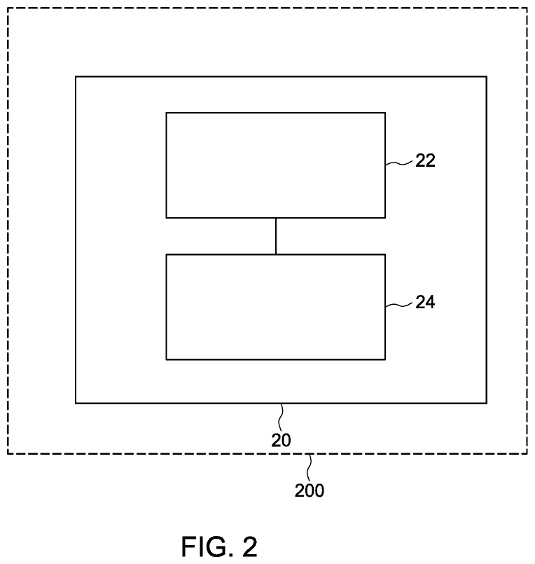

FIG. 2 shows block diagrams of an exemplary embodiment of a device 20 for a measuring device 200 or a ventilation device 300 and for determining a patient component of an airway flow of a ventilation gas of a patient being ventilated and of an exemplary embodiment of a measuring or ventilation device 200, 300. The device 20 comprises ole or more interfaces which are configured for the exchange of information with the measuring device 200 or with the ventilation device 300. The device 20 comprises a control unit, which is coupled with the one or more interfaces 22. The control unit 24 is configured to carry out one of the processes 10 being described here, to determine a time course of a respiratory muscle pressure of the patient, high-pass filtering of the time course of the respiratory muscle pressure in order to obtain a time course of a filtered respiratory muscle pressure and determination of the patient component of the airway flow based on the time course of the filtered respiratory muscle pressure.

FIG. 2 illustrates, moreover, an exemplary embodiment of a ventilation device 300 with a device 20 and an exemplary embodiment eta measuring device 200 with a device 20.

The device 20 comprises one or more interfaces 22, which are coupled with the control unit 24. The one or wore interfaces 22 may be configured, for example, in the form of a machine interface or 1n the form of a software interface. The one or more interfaces 22 may be configured in exemplary embodiments as typical interface(s) for communication in networks, or between network components or medical devices, e.g., ventilators, sensor units or measuring units, stimulators, etc. For example, they may be configured in exemplary embodiments by corresponding contacts. They may also be configured in exemplary embodiments as separate hardware and comprise a memory, which at least temporarily stores the signals to be transmitted or the received signals. The one or more interfaces 22 may be configured to receive electrical signals, for example, as a bus interface, as an optical interface, as an Ethernet interface, as a wireless interface, as a field bus interface, etc. They may, moreover, be configured in exemplary embodiments for wireless transmission and comprise a radio front end as well as corresponding antennas. Input and/or output devices, for example, display screen, keyboard, mouse, may also be connected via the one or more interlaces 22 in order to detect user inputs and or to make outputs possible.

The control unit 24 may comprise in exemplary embodiments one or more freely selectable controllers, microcontrollers, network processors, processor cores, such as digital signal processor cores (DSPs), programmable hardware components, etc.. Exemplary embodiments are not limited here to a particular type, of processor core. Any desired processor core or even a plurality of processor cores or microcontrollers are conceivable for implementing a control unit 24. Implementations in integrated form with other devices are also conceivable, for example, in a control unit, which additionally also comprises one or more other functions. A control unit 24 may be embodied in exemplary embodiments by a processor core, a computer processor core (CPU=Central Processing Unit), a graphics processor core (GPU=Graphics Processing Unit), an application-specific integrated circuit core (ASIC=Application-Specific Integrated Circuit), an integrated circuit (IC=Integrated Circuit), a one-chip system core (SOC=System on Chip), a programmable logic element or a field-programmable gate array with a microprocessor (FPGA=Field Programmable Gate Array) as a core of the component or components.

The present description uses the following terms and definitions:

### (Muscle) Breathing Effort:

The term is used in the generic sense. It designates the muscle effort of the patient to generate a respiratory muscle pressure in order to bring about a flow in the airway. If occlusion is performed during the breathing effort, i.e., &the airway how is interrupted by blockage, no flow develops in the airway, even though a respiratory muscle pressure (which can be measured as “mouth pressure” (pressure in the mouth area) if the airways are open) is generated. There is an isometric contraction of the respiratory muscles in this case. A physiological work always corresponds to the breathing effort, but the physiological work cannot be measured directly. A physical work, which is, by contrast, measurable, is only performed when the contraction is not generated isometrically, i.e., when a flow s generated in the airway.

### Breathing Assistance:

The breathing assistance is a ventilatory pendant to the muscle breathing effort. The ventilator assists the patients breathing effort detected (by triggering) synchronously with an assistance stroke. The patient consequently sets the breathing rhythm. It may be a pressure controlled assistance (the airway pressure is set) or—more rarely—a volume-controlled assistance (the breath volume is set). Physical work is performed by the ventilator in all cases, i.e., a part of the total work of breathing to be performed is taken from the patient.

### (Mandatory) Ventilation:

The total work of breathing is eliminated from the patient during mandatory ventilation. The breathing rhythm is determined by the machine. The patient is normally passive during the mandatory ventilation, so that there will be no conflict between the human/patient and the machine. The passivity of the patient is often brought about by sedatives and relaxants.

### WOB—Work of Breathing:

This is the physiological or physical work, which is performed for the breathing or/and ventilation. Even though no physical work is performed in case of isometric contraction in the sense of the kinetic equation, as it will be described below in the further course of the description, other definitions of work, e.g., the so-called pressure-time product (time integral), can be used.

### WOBtot—Total Work of Breathing:

This is the total physiological or physical work, which is performed for the breathing or/and ventilation.

### WOBvent—Machine/Ventilator-Side Work of Breathing:

This is the component of the work of breathing that is contributed by the ventilator.

### WORmus—Patient-Side (Muscle) Work of Breathing:

This is the component of the work of breathing that is performed solely by the patient both with and without stimulation of the muscles.

### WORspon—Spontaneous (Patient-Side) Work of Breathing

This is thea work of breathing performed by spontaneous intrinsic breathing of the patient. The muscles are not stimulated in the process.

### WOBstim—Stimulated (Patient-Side) Work of Breathing,:

This is the work of breathing performed by stimulation of the respiratory muscles of the patient.

### Pdrv—Driving Pressure:

Pdrv is the sum of the pressures that are generated by the ventilator and by the patient.

### Pvent—Ventlation Pressure:,

Pvent is the pressure that is generated by the ventilator.

Pmus—Muscle Pressure:

Pmus is the pressure that is generate(by the muscles of the patient alone, both with and without stimulation of the muscles.

### Pspon—Spontaneous Muscle Pressure:

Pspon is the component of the muscle pressure that is generated spontaneously, i.e., without stimulation, by the patient.

### Pstim—Stimulated Muscle Pressure:

Pstim is the component of the muscle pressure that is generated iy the patient solely based on the stimulation of the muscles.

### WOBbase and PmusBase—Basic Breathing Load:

The basic breathing load can be equated with the work of breathing or with the driving pressure, which is necessary to overcome the resistive, elastic and optionally other resistances and to reach a sufficient volume (e.g., the minute volume set by the clinical staff) in case of a healthy breathing pattern. The breathing, pattern is preferably assumed to be an energy optimised pattern. The work of breathing or the driving pressure can be generated on the patient side or land on the machine side. The latter would happen in case of a (mandatory) ventilation.

### Breathing Load:

The breathing load can be determined by detecting the work of breathing or the driving pressure, which work of breathing or driving pressure is actually generated. The breathing load is normally higher than the basic breathing load, since the breathing rhythm of the patient is not energy-optimized, e.g., the patient seeks to have a higher volume, e.g., based on shortness of breath than is needed or there is an asynchronism between the patient and the ventilator. The ventilator assumes a part of the breathing load in case of breathing assistance.

### Activation Signal:

This is a signal that detects the neuronal activation of the muscle (caused either by stimulation or by spontaneous breathing effort), e.g., the (s)EMG (surface electromyogram), EIM (electrical impedance myogram), MMG (mechanomyogram). As an alternative, signals that are detected by means of novel optical or acoustic (e,g., ultrasound) technology are also considered. The enveloping curve of the EMU will be used below as an activation signal (designated by “EMG” for simplicitys sake), without excluding other signals. It shall not be ruled out hereby that there are different activation signals of different muscle groups (e.g., diaphragm and intercostal muscles which yield an activation of their own each. The diaphragm activation signal is in the foreground in the context of the stimulation of the diaphragm (for example, by magnetic stimulation of the phrenic nerve).

### Activatability

This is the ability, electrically to stimulate (activate) the muscles, for example, by electrical or magnetic stimulation. The activation is preferably elicited by a so-called magnetic twitch stimulation, by a transient stimulation pulse with high intensity, which leads to a. maximum contraction of the stimulated muscle, as described, for example, in Walker, D. J.: “Prediction of the esophageal pressure by the mouth closing pressure following magnetic simulation of the phrenic nerve,” Dissertation University of Freiburg, 2006. Other stimulation patterns would be conceivable as well. The activation may be detected by means of an activation signal, preferably with EMG.

### Efficiency:

The efficiency is a pneumatic target variable (pressure or volume), which is achieved by a muscle activation. The “neuromechanical efficiency” (or “neuromuscular efficiency”) NME relates the muscle pressure produced and the “neuroventilatory efficiency” NVE relates the volume produced to the EMG, as is described, for example, in WO 2018143844, Liu et al.: “Neuroventiiatory efficiency and extubation readiness in critically ill patients,” Critical Care, 2012, as well as in Jansen D., et al,: “Estimation of the diaphragm neuromuscular efficiency index in mechanically ventilated critically ill patients,” Crifical Care, 2018. The determination of the efficiency often requires maneuvers, e.g., occlusions or changes in the breathing assistance. As is described there, the values have a diagnostic significance, e.g., in the assessment of the progression of the weaning from the ventilator.

### Maximum Possible Breathing Effort:

This corresponds to the work of breathing WOBmusMax, to the volume VolMusMax or to the muscle pressure PmusMax, which work of breathing, volume or muscle pressure can be achieved by maximum effort of the respiratory muscles. PmusMax is most likely to be able to be measured in a standardized manner. Thus, the value Plmax is frequently used in the literature as the indicator of the maximum possible muscle pressure, i.e., the maximum pressure produced during mouth closing. Since the voluntary contraction of the diaphragm leads to unreliable events, a so-called (usually magnetic) twitch stimulation, which can be per independently from the ability of the patient to cooperate, is frequently used recently to elicit the contraction. The PmusMax is normally detected by tt ea.€ts of an esophageal catheter, but the mouth closing pressure, which can be measured in a more simple manner, as it is described, for example, in Cattapan, S. E et al.: “Can diaphragmatic contractility be assessed by airway twitch pressure in mechanically ventilated patients?,” Thorax, 2003, has likewise proved to be meaningful. Triggering of the stimulation is advantageous here, as it is also described, for example, in Walker, D. J.; “Prediction of the esophageal pressure by the mouth closing pressure following magnetic stimulation of the phrenic nerve,” Dissertation, University of Freiburg 2006.

### LI Load (Load Index):

This is an indicator that depends on the ratio of the work of breathing produced to the maximum possible work of breathing, but the muscle pressure or another indicator may also be used instead of “work.” The load can be defined for the muscle pressure as

LI=Pmus/PmusMax.

### LBC—Load Bearing Capacity:

This is the ability of the muscles to exert by contraction a defined force or—in case of the respiratory muscles—pressure and thus to be able to perform work. To quantify the load hearing capacity, the muscle pressure/work of breathing produced by the soles is usually related to the muscle pressure/work of breathing that can be maximally produced. The load bearing capacity can thus be determined quantitatively as a function of the ratio of the basic breathing load to the maximum possible breathing effort. When the basic breathing load exceeds a defined part of the maximum possible breathing effort, the load bearing capacity is no longer present for exclusive spontaneous breathing and ventilation is mandatory. The load bearing capacity can be defined for the muscle pressure as

LBC−1−PmusBase/PmusMax.

### Exhaustion:

This happens after some time when the load bearing, capacity of the respiratory muscles is so low that the component contributed by the patient to the breathing load exceeds a defined part of the maximum possible breathing effort, e.g., 50%.

### Degree of Exhaustion:

The degree of exhaustion is linked with the load bearing capacity, with the breathing effort and with the duration hereof, but it cannot be calculated from these exclusively and directly, However, there are measured values, which can be calculated, for example, from the electromyogram of the muscles and can be used as a surrogate hr the degree of exhaustion; this is described, for example, in Kahl, L. et al,: “comparison of algorithms to quantify muscle fatigue in upper limb muscles based on sEMC1 signals,” Medical Engineering & Physics: 2016, as well as in DE 10 2015 011 390 A1.

The relationships of the relevant variables may also be described on the basis of formulas, some of which. will. be shown below.

The work of breathing can be calculated as an integral of the corresponding pressure relative to the volume, e.g., for the total work of breathing:

WOBtot=∫Pdrv(t)dV−∫Pdrv(t)·Flow(t) dt.

As an alternative (especially for the case of isometric load), the pressure-time product can be used:

WOBtot˜=∫Pdrv(t) dt.

The driving pressure is divided, analogously to the work of breathing, into different components:

Pdrv=Pvent+Pmus=Pvent+Pspon+Pstim WOBtot=WOBvent÷WOBmus=WOBvent+WOBspon+WOBstim.

The flow or the volume earn likewise be divided by calculation into the different components analogously to the work of breathing:

Flow=FlowVent+FlowMus=FlowVent+FlowSpon+FlowStim

and

Vol=VolVent+PMus=R·Flow+E·Vol+const

is valid for the basic kinetic equation at the breathing circuit, in which R and E=1/C are he breathing mechanical paras parameters resistance and elastance (the reciprocal value of compliance) of the patient. The equation is valid under the assumption that the respiratory system of the patient can be described as a simple RC module.

When the flow and the volume are defined (as described above) each as the sum of the components contributed by the ventilator and by the patient,

Pvent=R·FlowVent+E·VolVent+const

and

Pmus=R·FlowMus·E·VolMus+const.

are obtained for Pve it and Pmus.

The work of breathing of the ventilator and that of the patient can thus be calculated:

WOBvent=∫Pvent·Flow dt=∫Pdrv·FlowVent dt,

WOBmus=∫Pmus·Flow dt=∫Pdrv·FlowMus dt.

That there always are two possibilities can be detived from the kinetic equation (see above) and it can be proved by insertion into the integrals.

In a simplified hypothesis, Pmus can be assumed to be proportional to the EMG signal;

Pmus=NME·EMG

or, for example, as a linear combination of the EMG signals of two muscle groups

Pmus=NME1EMG1+NME2·EMG2,

wherein NME, NME1 and NME2 represent the neuromechanical efficiency of the respective muscle group. The kinetic equation

Pvent+Pmus=R·Flow+E·Vol+const

thus changes to

Pvent=R·Flow+E·Vol+const−NME EMG.

How NME can be determined is already described, for example, in DE 10 2007 062 214 B3, WO 2018 143 844 A, DE 10 2019 006 480 A1, US2021205561 (A1). The muscle activation is the sum of the spontaneous activity and of the activity triggered by stimulation

EMG=EMGspon+EMGstim,

wherein it is assumed that the amplitude of the activity triggered by stimulation is linked with the activatability k and with the amplitude of the stimulation intensity Istim\hat{ } in a multiplicative manner:

EMGstim\hat{ }=k·Istim\hat{ }.

The scalar relationship can then be used when indicators of the stimulation intensity and activation an Valid for broader time range.8 e.g., whole breaths, However, the stimulation takes place no typically as a sequence of weighted pulses with a distImee of 20-100 msec corresponding to 10-50 Hz (preferably 40-50 msec corresponding to 20-50 Hz). Each individual pulse (twitch) triggers an individual activation, but the breath-like shape of the activation signal is obtained only after the entire pulse sequence, the time course of the triggered activity EMGstim(t) differs markedly from the time course of the stimulation intensity Istim(t). The activatability can be represented as a simple constant (or characteristic) only in case of time-averaged variables. The kernel-based estimation is possible for the time characteristic, e.g., with the simple hypothesis

EMGstim(t)=Istim(t)*k(t),

in which * is the convolution symbol and k(t) is the core of the modeling (kernel) of the aetivatability, which core is to be estimated. Constant components (offsets) of the activation in the sense of a tonic muscle tension are ignored here. Istim(t) is usually a sequence of transient stimulation pulses. Then, k(t) corresponds to the pulse response of the activation to a stimulation pulse of the intensity Istim(t). There are many methods for estimating the kernel k(t), e.g., methods of system identification, stimulus-dependent averaging (for example, analogously to the peri-stimulus-time histogram) or least-squares estimation method. In the equation

EMG=EMGspon+Istim(t)* k(t),

EMGspon is assumed to be an unwanted signal, which is minimized by adaptation of the kernel. Finally, the spontaneous activity EMGspon can thus be determined as well, so that all factors of the kinetic equation.

Pvent(t)=R·Flow(t)+E·Vol(t)÷const−NME·[EMGspon(t)+k(t)*Istim(t)]

are known. The components of the total work of breathing and of the driving pressure can thus be determined and used for controlling the ventilation and the stimulation. Instead of estimating the sample values of the kernel, a parametric estimation may be carried out as well. The kernel could thus be considered to be a system pulse response and the parameters thereof could be identified. Correspondingly,

EMG=EMG1+EMG2 EMG1EMG1stim+EMG2spon÷EMG2stim and Pvent(t)=R·Flow(t)+E·Volt(t)+const

NME1[EMG1spon(t)+k1(t)* Istim1(t)]

NME2[EMG2spon(t)+k2(t)* Istim2(t)]

are valid for the activation of, e.g., two muscle groups possibly by means of stimulation.

The components of the work of breathing, which are produced by different muscle groups, can thus be determined. The specific stimulation makes possible especially stimulation maneuvers, which specifically affect defined muscle groups and lead to the activation. As a result, an estimation of the neuromeehanical efficiencies and of the ken Is or stimulation pulse responses is comparatively simple.

Instead of using work of breathing (WOB) or muscle pressure (Pmus) as target variables for the stimulation, it would also be possible, as at alternative, to use the component of the flow that is caused by the muscles, FlowMus. FlowMus or its integral over time, the volume VoiMus, may be advantageous under some circumstances, cf, US 2017 0252558 A1. It would be advantageous for this for the clinical staff to be very familiar with the terms flow and volume contrary to muscle pressure or work of breathing. The splitting of the flow or volume into patient and machine components is a basis in exemplary embodiments. When FlowMus is available, VolMus (as a time course), but also VTmus (tidal volume produced by muscles) or MVmus (minute volume contributed by muscles) can be determined in a very simple manner by an integration. These variables may be important for the respiratory diagnostics and therapy.

Exemplary embodiments can make it possible to calculate the patient component of the airway flow, for example, for triggering and automation during the ventilation. Exemplary embodiments can thus be divided, e.g., into two parts A and B, which are divided into a process for calculating the airway flow component and, the process 10 for determining a pattient component, the patient component being based directly on the spontaneous breathing activity. The use of exemplary. embodiments may-, however, differ markedly. Automation during the ventilation is involved in A, and the triggering and the cycling-off of assistance strokes is involved in B. The automation during ventilation is especially necessary in situations in which the patient displays spontaneous breathing efforts but is not able at the same time to ensure an adequate ventilation of the lungs. Processes for the automation therefore frequently employ indicators that detect the spontaneous breathing activity of the patient. Thus, some systems, for example, SmartCare of Dräger, etc., make use of the spontaneous respiration rate (fspn, spontaneous respiration rate), i.e., the number of breaths triggered by spontaneous breathing per minute. Furthermore, the spontaneous tidal volume (VT) achieved during the triggered breathing pressure assistance is used to adapt the degree of pressure assistance together with the end-tidal CO₂ value. Other processes likewise use fspn during the adaptation of the ventilation.

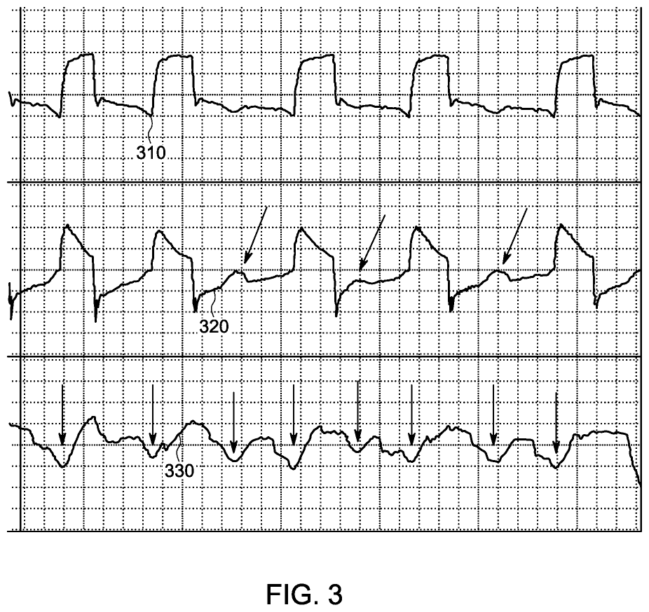

FIG. 3 shows a time course of a pressure 310 generated by a ventilator (top), a volume flow 320 between ventilator and patient (center) and a curve 330 of spontaneous breathing efforts of a patient (bottom). The true spontaneous respiration rate is higher by a factor of nearly 2 than the rate of the pressure-assisted strokes, cf. E10, which is suggested by the arrows at the respective curves. It is disadvantageous that the spontaneous respiration rate needed detects only the number of triggered breaths but not, e.g., all breathing efforts of the patient. For example. the so-called missed efforts are thus left out of consideration. These occur especially frequently when the pressure assistance is (excessively) high (see FIG. 3). Furthermore, it is a drawback that the achieved tidal volume (VT) depends directly on the degree of pressure assistance For example, a patient may breathe such that be triggers breaths with minimal energy. At the same time, a considerable spontaneous tidal volume is achieved based on the assistance, but this tidal volume is hardly correlated with the breathing effort, An automat on scheme, which will take into consideration the volume that the patient has reached solely based on his spontaneous breathing effort, cannot make use of the above-mentioned VT. An indicator of the respiratory muscle exertion can also be proposed or used for the control of the ventilation, especially for the triggering and the cycling-off, M addition to pneumatic signals (such as flow, pressure, volume). A so-called “improvised effort signal” is proposed in DE 100 2015 011 390 A1. A signal, which detects the activation of the diaphragin is used via an esophageal catheter in DE 10 2015 011 390 A1.

It is described in US2021205561 (A1) and DE 10 2007 062 214 B3 how a muscle pressure signal can be determined from the respiratory surface electromyogram. What is used in all these cases is an indicator of the muscle exertion or the respiratory drive. The muscle pressure signal Pmus can also be calculated if an estimate of the pleural pressure signal (detected by means of an esophageal catheter) and the estimate of the chest wall elastance are available. Triggering based on such indicators of the respiratory muscle exertion is difficult, because the signals are not equal to zero during the presumably passive phases (e.g., during passive exhalation), either. It is thus necessary to determine a threshold, which must, moreover, frequently be readjusted dynamically (on-line), because the activity level fluctuates. The start of the breathing effort is detected in case of an overshooting of this (possibly dynamic) threshold and a corresponding event is possibly elicited, for example, with the goal of triggering an assistance stroke. The cycling-off can be achieved analogously, but it then requires an additional (possibly dynamic) threshold. It is less problematic if a relative threshold (e.g., drop by x %) can be used for the cycling-off. The above-mentioned drawbacks can be avoided if the component of the airway flow (or the component of the volume integral of the flow), which is based directly on the spontaneous breathing activity, is determined. The measured airway flow also always contains the component of the flow that originates from the assistance by the ventilator. When a patient is being ventilated mechanically and is, in addition, breathing spontaneously, the flow of gas between the ventilator and the patient results from a superimposition of the mechanical ventilation and the spontaneous breathing. When the patient is inhaling, the gas flows from the ventilator to the patient. The volume Vol and the volume flow Vol′ are stated with a positive sign when the patient is inhaling, When the patient is exhaling, the gas flows from the patient to the ventilator (negative sign of flow/flow rates).

The following references will be used below, and the following variables are variable over time; as this is indicated in Table 1 below:

TABLE 1

| | |
|---|---|
| Vol_{ges}, | The total volume or the total volume flow, which flows from |
| Vol′_{ges} | the ventilator to the patient or in the opposite direction |
| Volₘᵤₛ, | The volume or the volume flow which the patient achieves |
| Vol′ₘᵤₛ | with his respiratory muscles, usually not measurable |
| | directly |
| Volᵥₑₙₜ, | The volume or the volume flow which the ventilator |
| Vol′ᵥₑₙₜ | achieves |
| P_{aw} | the pressure present at the ventilator |
| P_{drv} | The total pressure acting on the lungs (driving, pressure) |
| Pᵥₑₙₜ | The pressure generated by the ventilator |
| Pₘᵤₛ | The pressure which the patient generates with, his |
| | respiratory muscles |
| R, E | Resistance and elasticity of the respiratory system, |
| | comprising the lungs and the chest |
| PEEP | End-expiratory pressure |
| iPEEP | Intrinsic end-expiratory pressure, which remains in the |
| | lungs after a breath |
| τ, tau | Respiratory time constant of the respiratory system, |
| | which is considered to be a linear system |
| S | The frequency (complex number) |

The volume flow Vol'mus may also be triggered by a magnetically or electrically stimulated breathing of the patient. The tent “intrinsic breathing activity”of the patient designates the spontaneous breathing plus the stimulated breathing activity. The mechanical ventilation shall rather be synchronized with the nitrinsic breathing activity of the patient. The diagram in FIG. 3 illustrates a possible problem during mechanical ventilation. The curve at the very top in FIG. 3 shows the time course of the pressure generated by the ventilator, Pvent, and the curve in the center the volume flow between the ventilator and the patient, Vol'ges. The ventilator is controlled in one embodiment with the goal that the volume flow Vol'ges shall follow a predefined desired curve. The ventilator operates in this exemplary embodiment in a pressure-controlled manner, i.e., the now becomes established, and the pressure is predefined. As an alternative, it would also be possible to carry out the control the other way around, but this is rather not made use of during, spontaneous breathing, but only in case of mandatory ventilation. The lower curve shows the intrinsic breathing efforts of the patient, measured as esophageal pressure Pes. It can be seen that the spontaneous respiration rate is twice the frequency of the ventilation strokes. the ventilator is not synchronized optimally with the in insic breathing activity of the patient. An indicator of the breathing effort, the respiratory muscle pressure Pmus, directly describes the spontaneous breathing activity by, representing the spontaneous component. of the driving pressure. However, the direct correspondence with a corresponding flow (or volume) has not yet been known. It will be described below how the spontaneous component of the airway flow (called V′mus below) is derived and determined. It will then be shown how it is used in a meaningful manner. In the simple linear case, the kinetic equation for the breathing circuit is:

Paw+Pmus=R·V′+E·V+PEEP, (1)

in which the flow V′and the volume v are to be defined each as the sum of the components that is contributed by the patient and by the ventilator and PEEP is the positive end-expirator pressure, Paw is the pressure present at the ventilator, i.e.,

V′=V′mus+V′vent, and

V=Vmus+Vvent.

It is thus possible to write

Pmus=R·V′mus+E·Vmus (2)

and

V′vent=s/E·(Paw−PEEP)/(1+tau·s) (3)

is obtained after Laplace transformation in the frequency domain or

V′mus=S/E·Pmus/(1+tau·s) (4)

is Obtained in a preferred form depending on Pmus instead of Paw.

Here, tau=R/E is the respiratory time axis.

### Derivation:

$$V ’ = V ’ mus + V ’ vent$$

$$V ’ vent = V ’ m - V ’ mus$$

$$Pmus = R \cdot V ’ mus + E \cdot Vmus$$

$$Pmus = R \cdot V ’ mus + E / s \cdot V ’ mus = V ’ mus ( R + E / s )$$

$$\text{=>} V ’ mus = Pmus / ( R + E / s ) = s / E Pmus / ( 1 + tau \cdot s )$$

$$V ’ vent = V ’ - Pmus / ( R + E / s )$$

$$Paw + Pmus = R \cdot V ’ + E \cdot V + PEEP = R \cdot V ’ + E / s \cdot V ’ + PEEP = V ’ ( R + E / s ) + PEEP$$

$$\text{=>} {V}^{’} = 1 / ( R + E / s ) ( Paw + Pmus - PEEP )$$

$$\begin{matrix}V ’ vent = 1 / ( R + E / s ) ( Paw + Pmus ∼ PEEP ∼ Pmus ) \\ = 1 / ( R + E / s ) ( Paw ∼ PEEP ) \\ = 1 / ( E / s ( 1 + tau \cdot s ) ) ( Paw ∼ PEEP ) \\ = s / E \cdot 1 / ( 1 + tau \cdot s ) ( Paw ∼ PEEP ) .\end{matrix}$$

V′mus can consequently be calculated by means of a DT1 high-pass from Pmus. The process 10 may accordingly comprise a transformation of the time course of the respiratory muscle pressure into a frequency domain. For example, a Fourier transformation or a fast Fourier transformation (FFT) can be used for this purpose. The high-pass filtering can then be carried out in the frequency domain in order to obtain a filtered spectrum. The filtering in the frequency domain may be carried out, for example, by simply cutting off the, signal components in a lower frequency domain. The process 10 then comprises a transformation of the filtered spectrum into a time domain in order to obtain the time course of the filtered respiratory muscle pressure. This can be brought about analogously by using the correspondingly inverse transformation. The process 10 can also perform the high-pass filtering in exemplary embodiments in the time domain, which may have a favorable effect on the calculation times. The time domain processing may take place, for example, from one sample value to the next, whereas the frequency domain processing may require a block-by-block processing. For example, it is also possible to use a sliding Fourier transformation (Sliding DFT, which is based on a discrete Fourier transformation).

As to the device, the control unit 24 is configured to carry out the determination 12 of the time course of the respiratory muscle pressure on the basis of an electromyographic signal, of a pneumatic signal or of a mechanical signal. The determination 12 of the time course of the respiratory muscle pressure can be carried out on the basis of an electromyographic signal, of a pneumatic signal or of a mechanical signal. Combinations of these signals are conceivable as well; for example, Pmus can be calculated from Paw and Flow. For example, the parameters of the kinetic equation can be estimated in the time ranges in which the patient is passive (Pmus can consequently be assumed to be zero) and Pmus can then be calculated with the estimated parameters. The device 20 may correspondingly comprise electrodes for detecting the electromyographic signal, sensors for detecting a pneumatic signal or sensors for detecting a mechanical signal. Many different electrodes are conceivable now, and rt ton-invasive signal detection should be given preference. For example, electrodes attached to the skin may be used.

The control unit 24 may be configured, for example, to transform the time course of the respiratory muscle pressure itno a frequency domain and to carry out the high-pass filtering in the frequency domain in order to obtain a filtered spectrum and to transform the filtered spectrum into a time domain in order to obtain the time course of the filtered respiratory muscle pressure. Analogously to the process 10, the control unit 24 may also be configured to carry out the high-pass filtering in the time domain. The high-pass filtering may be carried out, for example, by means of differential equations, which are derived from the z or s transmission function and which can be solved with a comparatively low computing effort.

Only the respiratory time constant must be known for the time course (aside from a factor) as long as the respiratory system can be described sufficiently well enough by the kinetic equation

Pdrv=Pvent+Pmus=R*Flow+E′Vol+PEEP.

When more complicated respiratory systems are considered, e.g., patients with “baby lung” or pendelluft, then nonlinearities or additional time constants will appear, “Baby lung” means the state of an inelectatic lung, in which only a small region is open (can be ventilated), i.e., the lung acts during the ventilation as if it were the lung of a baby. The ventilatable volume will increase again only after opening the atelectatic region (in the sense of the so-called recruitment, especially by setting a sufficiently high PEEP). If atelectatic lungs are ventilated normally, the characteristic (volume per pressure) is highly nonlinear, so that the simple equation is no longer valid. The equation for the high-pass filtering would then be correspondingly different.

The idealized model can be used in practice as the starting point, because the determination of the parameters for more complex respiratory systems may be difficult and inaccurate and is also time-consuming. When the signal is also needed in its scaling (for example, when absolute volumes are to be determined by integration), an estimat on of the airway resistance or of the elastance can additionally be employed. Now, tan R can be determined in a more simple manner than E if tau is known in some exemplary embodiments.

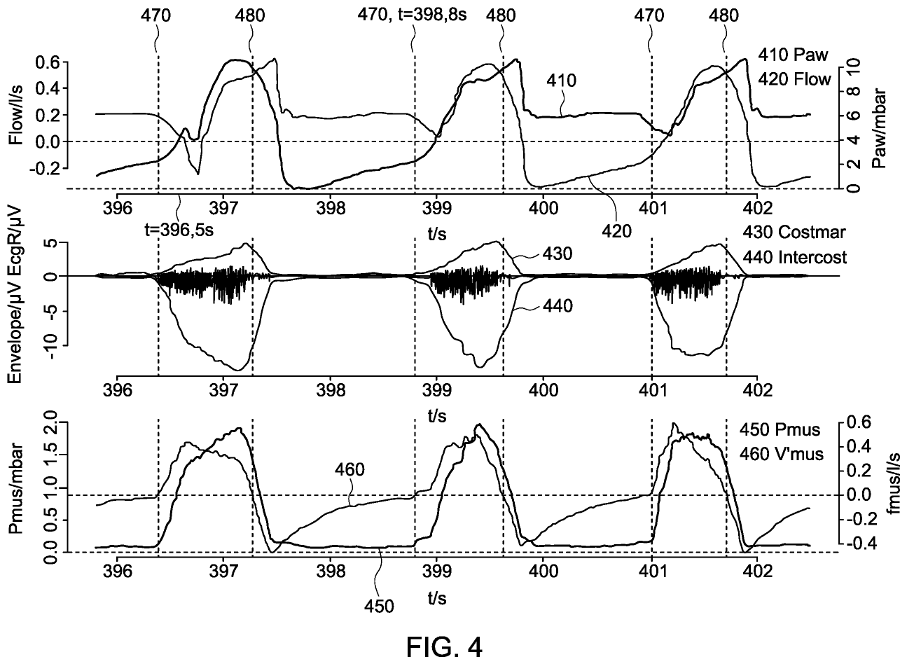

FIG. 4 shows examples of time courses of ventilation signals, FIG. 4 shows curves of Paw 410 and Flow 420 at the top, curves of the sEMG signals (Costmar 430 (signal at the lower costal arch) and Intercost 440 (signal of the intercostal muscle)) in the center and of the muscle signals Pmus 450 and V′mus=fasts 460 at the bottom.

The vertical broken lines 470 show the times at which the breathing effort begins, the lines 480 show the end of the breathing effort. They were determined directly from the V′mus signal 460 (zero crossing). The patient has COPD (chronic obstructive pulmonary disease) and suffers from “dynamic hyperinflation” or intrinsic PEEP. This is recognized from the fact that the Paw signal 410 drops long before the flow 420 has its zero crossing. The patient consequently must work hard to bring, the flow to zero from negative values (exhalation). A p0.1 occlusion is triggered at the time t=396.5 sec.

The diagram in FIG. 4 shows in the center the time e course of an electrical signal, which was genera ed by means of sEMG. Costmar 430 shows the time course that was obtained on the basis of two measuring electrodes close to the diaphragm. Intercost 440 shows the time course that was obtained on the basis of two electrodes at the level of the ribs (auxiliary respiratory muscles). For example, Rich a signal can be detected in the second intercostal space, counted from the top, to the left and right of the sternum (in order to have as little as possible into the region of the muscles of the chest and arm). The signal segments in the heartbeat time periods (ErgR=ECG removal) were removed in the two raw signals by calculation, which has led to gaps. The gaps were interpolated in the curves shown in the figures. The two solid lines (curves) are an indicator of the spontaneous breathing of the patient. Pmus is approximately equal in this case to Intercost with reversed sign (multiplied by (−1)). In general, Pmus is estimated depending on Costmar and intercost, for example, as described in E14. For example, Pmus is calculated from Costimar 430 and intercost 440 as a linear combination (with the. coefficients NNE1 and NME2).

For application B (triggering/cycling-off), the calculated V′mus can be treated in exactly the same manner as the flow V′during a flow triggering. What is ultimately involved here is the detection of the time at which the signal crosses the zero line, both for the triggering of a stroke and for the cycling-off it is not necessary to set a defined absolute threshold. This is due, among other things, to the fact that a possible offset is removed by the DT-1 high-pass filtering. An example of the curve of the V′mus signal 460 is shown in FIG. 4.

The process 10 may comprise in exemplary embodiments a determination of a time course of the patient component of the airway flow, as is shown, for example, in FIG. 4 on the basis of the signal curves for Pmus 450 and V′mus 460. The control unit 24 may be configured correspondingly in order to determine a tinge course of the patient component of the air ay flow.

As can be seen in. FIG. 4, the process 10 may comprise a determination of times 470 at which a breathing effort of the patient begins from the patient component 460 of the airway flow 420. The process 10 may also comprise an output of a trigger signal for a ventilator based on the times 470 at which a breathing, effort of the patient begins. The control unit 24 may correspondingly be configured to determine times 470 at which a breathing effort of the patient begins from the patient component of the airway flow and possibly to output a trigger signal for the ventilator based on the times at which a breathing effort of the patient begins.

Furthermore, the control unit 24 may be configured to determine times at which, a breathing effort of the patient ends from the patient component of the airway flow. The control unit 24 may be configured to output a cycling-off signal for a ventilator based on the times at which a breathing effort of the patient ends. This signal may be used, for example, for the ventilator to be able to end assistance strokes when the patient wants to exhale.

As is also shown in. FIG. 4, the process 10 may also comprise a determination of times 480 at which a breathing effort of the patient ends from the patient component 460 of the airway flow 420. It is then also possible to output a cycling-off signal for a ventilator based on the times at which a breathing effort of the patient ends. Moreover, the patient component of the airway flow may comprise a minute volume or a tidal volume, which is based on the spontaneous activity of the patient. Information on the minute volume or on the tidal volume can then be made available to a clinical staff.

For application A, automation of the ventilation, a substantially improved spontaneous respiration rate can be determined from the triggering process described by counting the number of detected efforts per minute. The process 10 can accordingly comprise a determination of a spontaneous respiration rate of the patient from the patient component of the airway flow. The control unit 24 may correspondingly be configured to determine a spontaneous respirationrate of the patient from the patient component of the airway flow.

It may be meaningful in this connection to take into consideration only efforts during which the spontaneous volume (Vmus) exceeds a meaningful minimum value, so that accidental fluctuations around the zero line are left out of consideration. The threshold necessary for this can be set in a comparatively simple manner, e.g., at a value between 10 mL and 50mL. The spontaneous volume can be calculated directly as an integral of V′mus 460 and it is thus likewise available for an automation. of the ventilation.

The process 10 may comprise in exemplary embodiments a determination of a high-pass characteristic for the high-pass filtering from the time course of the respiratory muscle pressure. As already explained, the high-pass characteristic may have been determined Z by a time constant tau. For example, the time constant tau may be determined from the time course of Pmus.

The time constant may also be calculated from the volume and the flow signal (an e curve is obtained during passive exhalation) and the quotient of the volume and flow yields the desired tau because of the derivation. As an alternative. R and E may be determined by means of regression (or another estimation method) and tau can be determined from, this. Pmus may also play an indirect role and the time ranges in which Pmus!=0 (Pmus not equal to 0) are not suitable for determining the parameter/parameters.

Knowledge of Pmus is required for the calculation of V′mus. However, if Pmus is known, both R and E, i.e, also the time constant tau=R/E, can be determined based on Equation (1), e.g., by means of linear regression, Kalman filtering or another estimation method. In addition or as an alternative, the sEMG signals can be used as indications or model signals for Pmus. If, e.g., sEMG=0, then Pmus is also zero and the regression can be calculated.

When sEMG!=0, i.e., “SEMCs is not equal to zero,” then sEMG can be included in the kinetic equation (simplest model hypothesis: Pmus=NME·EMG). If R and E or NME are then known, Pmus can be estimated, doing so either “pneumatically” (Pmus=R·Flow+E·Vol−Paw) or “electromyographically” (Pmus=NME·Flow) or as an averaging between them.

Accordingly, the high-pass characteristic can be determined on the basis of the kinetic equation for the breathing circuit of the patient. The process 10 may comprise the performance of a linear regression, of a Kalman filtering or of an estimation method based on the kinetic equation to determine the high-pass characteristic. A model of the lungs and chest can be taken into consideration now in interaction with components of the ventilator, of the ventilation tube system and of the patient-side coupling with the tube or mask or tracheostomy.

The control unit 24 may correspondingly be configured to determine a high-pass characteristic for the high-pass filtering from the time course of the respiratory muscle pressure, for example, in order to determine the high-pass characteristic on the basis of a kinetic equation for the breathing circuit of the patient. The control, unit 24 may be configured to carry out the linear regression, a Kalman filtering or an estimation method based on the kinetic equation to determine the high-pass characteristic. Estimation methods may comprise, for example, sequential Monte Carlo methods (particle filtering), nonlinear regression, Bayes estimator, etc.

In further exemplar embodiments, the time course of Pmus may also be determined by means of a probe in the esophagus, e,g. with the model equation:

Pmus=ECW·Vol−Pes. (1)

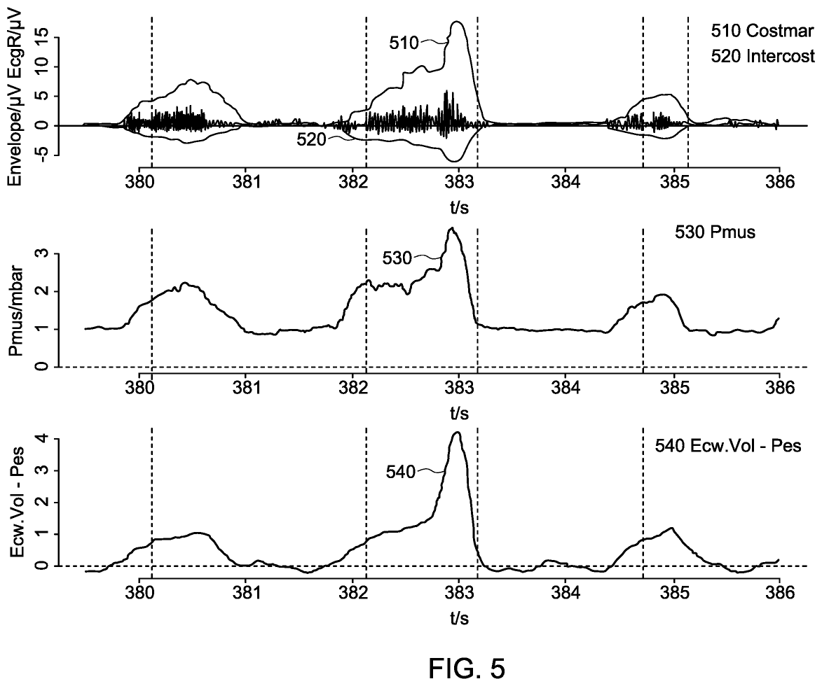

FIG. 5 shows further examples of time courses of ventilation signals. The diagram in FIG. 5 shows at the top another curve of the electrical signal, which was generated with two pairs of measuring electrodes (Costmar 510 and intercest 520). The curve in the center shows the curve for Pmus 530, which is equal to the curve of 510 Costmar. The curve at the bottom shows Ecw·Vol−Pes 540. This represents another type of estimation of Pmus, namely, an invasive signal detection by means of an esophageal sensor. The use of such invasive sensors (catheters) lay be carried out to create a reference for other estimations, especially those that are based on signals detected non-invasively. The reference of calculating Pmus via Pes can be considered to be a kind of standard. Therefore, this value is also called PmusRef. Since the restoring force of the chest (chest wall) is to be taken into consideration, Pes=ECW·Vol−Pmus is true.

Exemplary embodiments create, for example, a way of detecting automatically when the patient begins to make an intrinsic breathing effort. Now,

Volges=Volmus+Volvent (2)

Vol′ges=Vol′mus+Vol′vent (3)

Pdrv=Pmus+Pvent. (4)

When the ventilator is connected. then

Paw=Pvent (5)

where “aw” is the abbreviation for “airway,”

Pdrv is always the sum of all driving pressures (as in Equation (4)),

If Pmus=0, then

Paw=Pvent=Pdrv.

A simple lung mechanical model is:

$$Pdrv = Pmus + Pvent = R \cdot Vol ’ ges + E \cdot Volges + PEEP . \qquad ( 6 )$$

The ventilation strokes expand the lungs and overcome the resistance. Therefore,

Pvent=R·Vol′vent+E·Vol vent+PEEP. (7)

Hence,

Pmus=R·Vol′mus+E·Volmus. (8)

in addition; τ=R/E.

The LaPlace transformation yields in the frequency domain

$$Vol ’ mus = Vol ’ ges - Vol ’ vent = Vol ’ ges - s / E * ( Pvent - PEEP ) / ( 1 + τ \cdot s ) . \qquad ( 9 )$$

Then follows:

Vol′mus=s/E·Pmus/(1+τ·s). (10)

The volume flow Vol′mus generated by, the patient can consequently be calculated according to formula (10) if the respiratory time constant τ is known. For example, a DT1 high-pass filter is used. A corresponding transformation may also be applied to Pvent and a rule for Vol′vent will then be obtained. A DT1 high-pass filter then yields.

Vol′vent=s/E·(Pvent−PEEP)/(1+τ·s). (11)

A possible improvement, which is achieved by the present invention, is illustrated in the diagram in the top part of FIG. 4. The Flow 420 is the time course of the volume flow Vol′ges [L/sec], wherein segments above the zero line designate an inhalation process. Paw 410 is the time course of the pressure Pvent in [mbar}, which the ventilator achieves and which is always greater than or equal to zero, especially when PEEP >0 is set. Each line 470 shows the start of an attempt of the patient to inhale air. When PEEP=0 and the patient is inhaling intensely, the airway pressure may briefly collapse when the ventilator is unable to resupply a sufficient quantity of flow. Then, Pvent <0 could become true. The ventilation strokes are triggered in the example being shown by the curve of Pmus 450, which is determined, for example, on the basis of one or more sEMG signals, see above. The ventilation strokes lag behind the spontaneous breathing of the patient, which is suggested by the vertical lines 470. This can be seen best for t=398.8. The patient is already attempting. to inhale air.

Since Flow<0, the ventilator does not yet detect the inhalation effort. The ventilator can trigger only when the condition is met (t=399) and respond with an assistance stroke. Only the PEEP controller is running before that, i.e., the ventilator is trying to resupply Flow as best as it can in order to maintain the PEEP at a constant level. However, it cannot succeed in view to the enormous breathing effort of the patient, which is manifested in the collapse of the Paw pressure between T=398.8 and t=399.

Consequently, the ventilator is at first always still in the phase of exhalation and attempts to maintain the PEEP, When the patient now wants to inhale, then the ventilator slightly adjusts the flow, but it does not lag behind. Therefore, the pressure collapses slightly. The patient does not have to breathe against the device, and the device just simply does not assist. This happens only, when the flow sensor has detected a positive Flow. The process is then switched over into the phase of inhalation and a pressure stroke is triggered. The Flow developing now originates (to a low extent) from the patient, who has triggered, and mostly from the ventilator, which has responded with the stroke. If the ventilation strokes were ideally synchronized with the spontaneous breathing of the patient, the pressure would already have to rise at the lines 470, and in practice shortly thereafter. The ventilator ideally supplies the volume such that a set assistance pressure will be reached as rapidly as possible. A zero crossing of V′mus (start of inhalation) consequently triggers a ventilation stroke. Exemplary embodiments thus make a better synchronization possible. In particular, the start and the end of a ventilation stroke are better synchronized with the spontaneous breathing activity of the patient, both in terms of the time and in terms of the intensity.

The contribution of the spontaneous breathing to the minute volume Vol′ges is detected in further exemplary embodiments.

The diagram in FIG. 4 (bottom) shows the time course of the pressure Pmus 450 and that of the volume flow Vol′mus 460, which the spontaneous breathing of the patient achieves. The lines 470 indicate when the patient begins to inhale. The respective next zero crossing of Vol′mus 480 indicates when the patient begins to exhale. The pressure Pmus drops already before the start of the exhalation.

The aspects and features that are described in connection with one or more of the examples and figures described in detail above may also be combined with one or more of the other examples in order to replace an identical feature of the other example or in order to additionally introduce the feature into the other example.

Examples may, furthermore, be or pertain to a computer program with a program code (provided as a non-transitory, tangible medium (or media)) for executing cane or more of the above processes when the computer program is executed on a computer or on a processor. Steps, operations or processes of different processes described above may be executed by programmed computers or processors. Examples may also cover program memory devices, e.g., digital storage media, which are machine-readable, processor-readable or computer-readable and code machine-executable, processor-executable or computer-executable programs of instructions. The instructions execute some or all of the steps of the above-described processes or cause them to be executed. The program memory devices may comprise or be, for example, digital memories, magnetic storage media, for example, magnetic disks and magnetic tapes, hard drives or optically readable digital storage media. Further examples may also cover computers, processors or control units, which are programmed to execute the steps of the above-described processes, or (field)-programmable logic arrays ((F)PLAs=(Field) Programmable Logic Arrays) or (field)-programmable gate arrays t(F)PGA=(Field) Programmable Gate Arrays), which are programmed to execute the steps of the above-described processes.

Only the principles of the disclosure are represented by the description and the drawings. Furthermore, all the examples mentioned here shall be used, in principle, expressly only for purposes of illustration in order to support the reader in understanding the principles of the disclosure and of the concepts contributed by the inventor (inventors) to the farther development of the technology. All the statements made here about principles, aspects and examples of the disclosure as well as concrete examples thereof comprise equivalents thereof.

A function block designated as “means for . . . ” performing a certain function may pertain to a circuit, which is configured for performing a certain function. Thus, a “means for something” may be implemented as a “means configured for something or suitable for something,” e.g., a structural component or a circuit configured for or suitable for the respective task.

Functions of different elements shown in the figures, including those of each function block designated as “means,” means for providing a signal,” “means for generating a signal,” etc., may be implemented in the form of dedicated hardware, e.g., “a signal provider,” “a signal processing unit,” “a processor,” “a control,” etc., as well as as hardware capable of executing software in connection with corresponding software. In case of provision by a processor, the functions may be provided by an individual dedicated processor, by an individual, jointly used processor or by a plurality of individual processors, some of which or all of which may be used jointly. However, the term “processor” or “control” is far from being limited to hardware capable exclusively of executing software, but it ma comprise digital signal processor hardware (DSP hardware; DSP=Digital Signal Processor), network processor, application-specific integrated circuit (ASIC=Application Specific integrated Circuit), field-programmable logic array (FPGA=Field Programmable Gate Array), read-only memory (ROM=Read Only Memory) for storing software, random access memory (RAM=Random Access Memory) and non-volatile storage device (storage). Other hardware, conventional and or customer-specific, may be included as well.

A block diagram may represent, for example, a schematic circuit diagram, which implements the principles of the disclosure. Similarly, a flow chart, a flow diagram, a state transition diagram, a pseudocode and the like may represent different processes, operations or steps, which are represented, for example, essentially in computer-readable medium and are thus executed by a computer or processor, regardless of whether such a computer or processor is explicitly shown. Processes disclosed in the description or in the patent claims may be implemented by a component, which has means for carrying out each and every one of the respective steps of these processes.

It is apparent that the disclosure of a plurality of steps, processes, operations or functions disclosed in the description or in the claims shall not be interpreted as being arranged in the defined order, unless this is explicitly or implicitly stated otherwise, e.g., for technical reasons. Therefore, these are not limited by the disclosure of a plurality of steps or functions to a defined order, unless these steps or functions are not replaceable for technical reasons. Further, an individual step, function, process or operation may include in some examples a plurality of partial steps, partial functions, partial processes or partial operations and/or be broken up into these. Such partial steps may be included and be a part of the disclosure of this individual step, unless they are explicitly excluded.

Furthermore, the following embodiments are hereby included in the detailed description, in which each embodiment can stand by itself as a separate example. While each embodiment can stand by itself as a separate example, it should be noted that, even though embodiments that reference other embodiments may pertain in the embodiment to a defined combination with one or more other embodiments, other examples may also comprise a combination of the referencing embodiment with the subject of every other the referencing embodiment or the non-referencing embodiment. Such combinations are explicitly proposed here, unless it is stated that a defined combination is not intended. Furthermore, features of a referencing embodiment shall also be included for every other non-referencing embodiment, even if this referencing embodiment does not explicitly reference the other non-referencing embodiment.

Further and preferred embodiments of the present. invention with a concept for determining a patient component of an airway flow of a ventilation gas of a patient being ventilated will be described in more detail below with respect to a concept of determining the patient component of the airway flow based on the time course of a pressure parameter or of a respiratory muscle pressure.

A basic embodiment includes a process for determining a patient component of an airway flow of a ventilation gas of a patient being ventilated, with
determination of a time course of a respiratory muscle pressure of the patient,
high-pass filtering of the time course of the respiratory muscle pressure in order to obtain a time course of a filtered respiratory muscle pressure, and
determination of the patient component of the airway flow based on the time course of the filtered respiratory muscle pressure.

A preferred embodiment based on the basic embodiment may also comprise a transformation of the time course of the respiratory muscle pressure into a frequency domain, wherein the high-pass filtering is carried out in the frequency domain in order to obtain a filtered spectrum.

A preferred embodiment based on at least one of the above-described embodiments may also comprise a transformation of the filtered spectrum into a time domain in order to obtain the time course of the filtered respiratory muscle pressure.

In a preferred embodiment based on at least one of the above-described embodiments, the high-pass filtering may be carried out in the time domain.

In a preferred embodiment based on at least one of the above-described embodiments, the patient component of the airway flow comprises a minute volume or a tidal volume, which is based on the spontaneous activity of the patient.

In a preferred embodiment based on at least one of the above-described embodiments, the determination of the time course of the respiratory muscle pressure is carried out on the basis of an electromyographic signal, of a pneumatic signal or of a mechanical signal.

In a preferred embodiment based on at least one of the above-described embodiments, the determination of i high-pass characteristic is carried out for the high-pass filtering from the time course of the respiratory muscle pressure.

In a preferred embodiment based on a at least one of the above-described embodiments, the high-pass characteristic is determined by a time constant.

In a preferred embodiment based on at least one of the above-described embodiments, the determination of the high-pass characteristic is based on a kinetic equation for the breathing circuit of the patient.

In a preferred embodiment based on at least one of the above-described embodiments, the determination of the high-pass characteristic based on the kinetic equation comprises the performance of a linear regression, of a Kalman filtering or of an estimation method.

A preferred embodiment based on at least one of the above-described embodiments comprises a determination of a time course of the patient component of the airway flow.

A preferred embodiment based on at least one of the above-described embodiments comprises a determination of times at which a breathing effort of the patient begins from the patient component of the airway flow.

A preferred embodiment based on at least one of the above-described embodiments comprises an output of a trigger signal for a ventilator based on be times at which a breathing effort of the patient begins.

A preferred embodiment based on at least one of the above-described embodiments comprises a determination of times at which a breathing effort of the patient ends from the patient component of the airway flow.

A preferred embodiment based on at least one of the above-described embodiments may also comprise an output of a cycling-off signal for a ventilator based on the times at which a breathing effort of the patient ends.

A preferred embodiment based on at least one of the above-described embodiments may also comprise a determination of a spontaneous respiration rate of the patient from the patient component of the airway flow.

A basic embodiment may comprise a computer program with a program code for carrying out at least one of the above-described embodiments. The program code may advantageously be executed on a computer, on a processor or on a programmable hardware component.

A basic embodiment includes a device for a measuring device or a ventilation device and for determining a patient component of an airway flow of a ventilation gas of a patient being ventilated, with one or more interfaces, which are configured for the exchange of information with the measuring device or with the ventilation device, and with a control unit, which is configured
to determine a time course of a respiratory muscle pressure of the patient,
to perform a high-pass filtering of the time course of the respiratory muscle pressure in order to obtain the time course of a filtered respiratory muscle pressure, and
to determine the patient component of the airway flow based on the time course of the filtered respiratory muscle pressure.

In a preferred embodiment based on at least one of the above-described embodiments, the control unit may be configured to carry out the determination of the time course of the respiratory muscle pressure on the basis of an electromyographic of a pneumatic signal or of a mechanical signal.

In a preferred embodiment based on at least one of the above-described embodiments, the device may also comprise electrodes for detecting the electromyographic signal, sensors for detecting a pneumatic signal or sensors for detecting a mechanical signal.

In a preferred embodiment based on at least one of the above-described embodiments, the control unit may be configured to transform the time course of the respiratory muscle pressure into a frequency domain and to carry out the high-pass filtering in the frequency domain in order to obtain a filtered spectrum.

In a preferred embodiment based on at least one of the above-described embodiments, the control unit may be configured to transform the filtered spectrum into a time domain in order to obtain the time course of the filtered respiratory muscle pressure.

In a preferred embodiment based on at least one of the above-described embodiments, the control unit may be configured to carry out the high-pass filtering in the time domain.

n a preferred exemplary embodiment based on at least one of the above-described embodiments, the patient component of the airway flow can also comprise a minute volume or a tidal volume, which is based on the spontaneous activity.

In a preferred embodiment based on at least one of the above-described embodiments, the control unit may be configured to determine a high-pass characteristic for the high-pass filtering from the time course of the respiratory muscle pressure. The high-pass characteristic may have been determined by a time constant.

In a preferred embodiment based on at least one of the above-described embodiments, the control unit may be configured to determine the high-pass characteristic based on a kinetic equation for the breathing circuit of the patient.

In a preferred embodiment based on at least one of the above-described embodiments, the control unit may be configured to perform a linear regression, a Kalman filtering or an estimation method based on the kinetic equation to determine the high-pass characteristic.

In a preferred embodiment based on at least one of the above-described embodiments, the control unit is configured to determine a time course of the patient component of the airway flow.

In a preferred embodiment based on at least one of the above-described embodiments, the control unit may be configured to determine times at which a breathing effort of the patient begins from the patient component of the airway flow.

In a preferred embodiment based on at least one of the above-described embodiments, the control unit may be configured to output a trigger signal for the ventilator based on the times at which a breathing effort of the patient begins.

In a preferred embodiment based on at least one of the above-described embodiments, the control unit may be configured to determine times at which a breathing effort of the patient ends from the patient component of the airway flow.

In a preferred embodiment based on at least one of the above-described embodiments, the control unit may be configured to output a cycling-off signal for the ventilator based on the times at which a breathing effort of the patient ends.

In a preferred embodiment based on at least e of the above-described embodiments, the control unit may be configured to determine a spontaneous respiration rate of the patient from the patient component of the airway flow.

A measuring device and/or a ventilation device may be configured on the basis of at least one of the above-described embodiments of the device.

Table 2 below comprises the abbreviations and terms used within the framework of the present invention along with respective brief explanations.

TABLE 2

| Symbol | Description/Brief explanation | Units |
|---|---|---|
| R | Resistance of the respiratory system; | mbar/(L/sec) |
| | R = P/(dV/dt) | |
| C | Compliance; C = V/P | L/mbar |
| E | Elasticity of the respiratory system; | mbar/L |
| | E = I/C; E = P/V | |
| τ, tau | Time constant of the respiratory system | sec |
| S | Frequency (complex number) | s⁻¹ |
| Flow, | Airway gas flow, flow rate of gases, | L/min, |
| dV/dt | which flows away from the patient or | L/sec |
| | flows towards the patient | |
| Vol, V | Volume, which flows from/to the patient. | L |
| sEMG, | EMG signal, time course of the EMG signal | μV |
| sEMG(t) | | |
| MV | Minute volume | L |
| V_{T} | Tidal volume of the patient | L |
| Vol′_{ges} | Total volume flow, which flows from/to | L/min, |
| | the patient | L/sec |
| Volₘᵤₛ | Volume caused by muscle activity | L |
| Vol_{musMax} | Maximum volume caused by muscle activity | L |
| Vol′ₘᵤₛ | Volume flow caused by muscle activity | L/sec |
| Flowₛₚₒₙ | Breathing gas flow caused by spontaneous | L/min, |
| | muscle activity | L/sec |
| Flowₘᵤₛ | Volume flow caused by muscle activity | L/min, |
| | | L/sec |
| Volᵥₑₙₜ | Volume produced by the ventilator | L |
| Vol′ᵥₑₙₜ | Breathing gas flow produced by the | L/min, |
| | ventilator | L/sec |
| Volₛₜᵢₘ | Volume produced by stimulation | L |
| V_{T mus} | Tidal volume produced by muscle activity | L |
| MVₘᵤₛ | Minute volume produced by muscle activity | L |
| P_{aw} | Airway pressure | mbar |
| P_{drv} | Total pressure acting on the respiratory | mbar |
| | system (driving pressure) | |
| Pᵥₑₙₜ | Ventilation pressure, the pressure | mbar |
| | generated by the ventilator | |
| Pₘᵤₛ, | Pressure caused by muscle activity | mbar |
| Pₘᵤₛ(t) | | |
| P_{mus base}, | Basic breathing load, the minimum pressure | mbar |
| P_{base} | that is necessary to overcome the | |
| | respiratory resistances | |
| P_{musMax} | Pressure caused by maximum muscle activity | mbar |
| PIₘₐₓ | Maximum pressure generated during | mbar |
| | inhalation with the month closed | |
| Pₛₚₒₙ | Spontaneous muscle pressure, pressure | mbar |
| | caused by spontaneous muscle activity | |
| Pₛₜᵢₘ | Stimulated muscle pressure, pressure | mbar |
| | caused by muscle stimulation | |
| P_{insp.} | Airway pressure during the phase of | mbar |
| | inhalation (inspiration) | |
| P_{exp.} | Airway pressure during the phase of | mbar |
| | exhalation (expiration) | |
| PEEP | Positive end-expiratory pressure at the | mbar |
| | end of the phase of exhalation | |
| iPEEP | Intrinsic end-expiratory pressure | mbar |
| WOB | Work of Breathing; | Nm, J |
| | [mbar * L] = [1 hPa * m³/1000] = 0.1 Nm | |
| WOBₗₒₜ | Total work of breathing | Nm, J |
| WOBᵥₑₙₜ | Work of breathing produced by the | Nm, J |
| | ventilator | |
| WOBₛₚₒₙ | Work of breathing produced by spontaneous | Nm, J |
| | muscle activity | |
| WOBₛₜᵢₘ | Work of breathing produced by stimulation | Nm, J |
| WOB_{base} | Basic breathing load in the senses of work | Nm, J |
| | of breathing | |
| WOB_{musMax} | Work of breathing produced by maximum | Nm, J |
| | muscle activity | |
| NVE | Neuroventilatory efficiency | L/μV |
| NME | Neuromechanical efficiency | mbar/μV |
| LBC | Load Bearing Capacity | — |
| LI | Load Index | — |
| EMGₛₜᵢₘ | EMG signal brought about by stimulation | μV |
| EMGₛₚₒₙ | EMG signal brought about by spontaneous | μV |
| | activity | |
| SVE(t) | Ventilatory efficiency relative to muscle | L/% |
| | stimulation | |
| | SVE(t) = k(t) · NVEstim | |
| k(t) | Activatability | μV/% |
| NVEₛₜᵢₘ | Neuroventilatory efficiency during | L/μV |
| | stimulation | |
| NMEₛₜᵢₘ | Neuromechanical efficiency during | mbar/μV |
| | stimulation | |
| NMEₛₚₒₙ | Neuromechanical efficiency during | mbar/μV |
| | spontaneous breathing/spontaneous | |
| | muscle activity | |
| NVEₛₚₒₙ | Neuroventilatory efficiency during | L/μV |
| | spontaneous breathing/spontaneous | |
| | muscle activity | |
| Iₛₜᵢₘ, | Stimulation intensity, intensity of the | — |
| Iₛₜᵢₘ(t) | stimulation signal | |
| a2 | Component of driving pressure | — |
| b2 | Component of driving pressure | — |
| A | Positive feedback factor | — |
| Beta | β = α/(1 + α) | — |

All the patent documents and publications along with publication numbers and with short titles of the publications are listed in Table 3 below. The full titles can be found in the explanations on the state of the art in the introduction to the specification. The reference numbers [E1] through [E38] listed in this table are used at times in the specification.

TABLE 3

| [Ref] | Publication/disclosure/brief title |
|---|---|
| [E1] | U.S. Pat. No. 5,820,560 |
| [E5] | WO2019154834 |
| [E6] | WO2019154837 |
| [E7] | WO2019154839 |
| [E8] | WO2020079266 |
| [E9] | DE102019006480 |
| [E11] | Walker, D. J.: “Prediction of the esophageal pressure . . .” |
| [E13] | US2017/0252558 |
| [E14] | DE102019006480 |
| [E15] | DE102019007717 |
| [E16] | US2021205561 (A1) (DE102020000014) |
| [E17] | DE102007062214 |
| [E18] | WO2018143844 |
| [E19] | Kahl, L. et al.: “Comparison of algorithms to . . .” |
| [E20] | DE102015011390 |
| [E21] | Jansen D. et al.: “Estimation of the diaphragm |
| | neuromuscular . . .” |
| [E22] | Liu L. et al.: “Neuroventilatory efficiency and extubation |
| | readiness . . .” |
| [E23] | Cattapan, S. E. et al.: “Can diaphragmatic contractility |
| | be assessed by . . .” |
| [E24] | Younes, M. et al.: “A method for monitoring and |
| | improving . . .” |
| [E25] | Putensen C. et al.: “Long-term effects of spontaneous |
| | breathing . . .\| |
| [E26] | Bellani, G. et al.:” “The Ratio of Inspiratory Pressure |
| | Over . . .” |
| [E27] | U.S. Pat. No. 7,021,310 |
| [E28] | US20120103334 |
| [E30] | Otis, A. B. et al.: “Mechanics of Breathing in Man” |
| [E31] | US20150366480 |
| [E32] | WO2020188069 |
| [E34] | US 2022 072249 (A1) (DE102020123138) |
| [E35] | Shalaby, R. E. -S.: “Development of an Electromyography |
| | Detection System . . .” |
| [E36] | Osuagwu, B. A. C. et al.: “Active proportional |
| | electromyogram controlled . . .” |
| [E37] | US2009114224 (A1) (DE102007052897) |
| [E38] | Virtanen, J. et al.: “Instrumentation for the measurement |
| | of electric . . .” |

### LIST OF REFERENCE NUMBERS

10 Process for determining a patient component of an airway flow of a ventilation gas of a patient being ventilated
12 Determination of a time course of a respiratory muscle pressure of the patient
14 High-pass filtering oldie time course of the respiratory muscle pressure to obtain a time course of a filtered respiratory muscle pressure
16 Determination of the patient component of the airway flow based on the time course of the filtered respiratory muscle pressure
20 Device for a measuring device or for a ventilation device and for determining a patient component of an airway flow of a ventilation gas of a patient being ventilated
22 One or more interfaces
24 Control unit
100 Measuring device
300 Ventilation device
310 Time course of a pressure generated by a ventilator
320 Volume flow between ventilator and patient
330 Cur e describing spontaneous breathing efforts of a patient
410 Curve describing Paw
420 Curve describing Flow
430 Costmar
440 Intercost
450 Curve describing Puns
460 Curve describing V′mus=fnus
470 Times at which the breathing effort begins
480 Times at Which the breathing effort ends
510 Costmar
520 Intercost
530 Pmus
540 Ecw·Vol−Pes

What is claimed is:

1. A device for determining a patient component of an exchange of gas of a patient being ventilated, for a measuring device or a ventilation device, the device comprising:
an interface configuration comprising one or more interfaces configured for an exchange of information with the measuring device or with the ventilation device;
a control unit configured:
to determine a time course of a respiratory muscle pressure of the patient;
to carry out a high-pass filtering of the time course of the respiratory muscle pressure to obtain a time coarse of a filtered respiratory muscle pressure; and
to determine the patient component based on the time course of the filtered respiratory muscle pressure.

2. A device in. accordance with claim 1, wherein the control unit is configured to carry out the determination of the time course of the respiratory muscle pressure based on an electromyographic signal, a pneumatic signal or a mechanical signal.

3. A device in accordance with claim 1, wherein the control unit is configured to carry out the high-pass filtering in the time domain in order to obtain the time course of the filtered respiratory muscle pressure.

4. A device in accordance with claim 1, wherein the control unit is configured:
to carry out a transformation of the time course of the respiratory muscle pressure into the frequency domain;
to carry out the high-pass filtering in the frequency domain in order to obtain a filtered spectrum; and
to carry out a transformation of the filtered spectrum into the time domain in order to obtain the tin course of the filtered respiratory muscle pressure.

5. A device in accordance with claim 1, wherein the patient component of the exchange of gas comprises a minute volume or a tidal volume, which is based on the spontaneous activity of the patient.

6. A device in accordance with claim 1, wherein the control unit is configured to determine a high-pass characteristic for the adapted performance of the high-pass filtering based on at least one of:
the time course of the respiratory muscle pressure; and
a kinetic equation for the breathing circuit of the patient.

7. A device in accordance with claim 6, wherein the control unit is configured to perform a linear regression, a Kalman filtering or an estimation based on the kinetic equation to determine the high-pass characteristic.

8. A device in accordance with claim 1, wherein at least one of the control unit is configured to determine times at which a breathing effort of the patient ends from the patient component; and
the control unit is configured tip determine times at which a breathing effort of the patient begins from the patient component.

9. A device in accordance with claim 8, wherein at least one of:
the control unit is configured to output a trigger signal for the ventilator, and
the control unit is configured to output a cycling-off signal for the ventilator based on times at which a breathing effort of the patient begins.

10. A device in accordance with claim 1, wherein the control unit is configured to determine a spontaneous respiration rate of the patient from the patient component.

11. A device in accordance with claim 2, further comprising:
electrodes for detecting the electromyographic signal:, or
sensors for detecting the pneumatic signal; or
sensors for detecting the mechanical signal.

12. A measuring device or ventilation device comprising:
a device for determining a patient component of an exchange of gas of a patient being ventilated, the device comprising:
an interface configuration comprising one or more interfaces configured for an exchange of information with the measuring:, device or with the ventilation device;
a control unit configured:
to determine a time course of a respiratory muscle pressure of the patient;
to carry out a high-pass filtering of the time course of the respiratory muscle pressure to obtain a time course of a filtered respiratory muscle pressure; and
to determine the patient component based on the time course of the filtered respiratory muscle pressure.

13. A measuring device or ventilation device in accordance with claim 12, wherein the control unit is configured to carry out determination of the time course of the respiratory muscle pressure based on an electromyographic signal, a pneumatic signal or a mechanical signal.

14. A measuring device or ventilation device in accordance with claim 12, wherein the control unit is configured to carry out the high-pass filtering in the time domain in order to obtain the time course of the filtered respiratory muscle pressure.

15. A measuring device or ventilation device in accordance with claim 12, wherein the control unit is configured:
to carry out a transformation of the time course of the respiratory muscle pressure into the frequency domain;
to carry out the high-pass filtering in the frequency domain in order to obtain a filtered spectrum; and
to carry out a transformation of the filtered spectrum into the time domain in order to obtain the time course of the filtered respiratory muscle pressure.

16. A measuring device or ventilation device in accordance with claim 12, wherein the patient component of the exchange of gas comprises a minute volume or a tidal volume, which is based on the spontaneous activity of the patient.

17. A measuring device or ventilation device in accordance with claim 12, wherein the control unit is configured to determine a high-pass characteristic for the adapted performance of the high-pass filtering based on at least one of:
the time course of the respiratory muscle pressure; and
a kinetic equation for the breathing circuit of the patient.

18. A process for determining a patient component of a gas exchange of a patient being ventilated, the process comprising the steps of:
detecting and determining a time course of a respiratory muscle pressure of the patient based on an electromyographic signal, or a pneumatic signal or a mechanical signal;
performing an adapted high-pass filtering for the time course of the respiratory muscle pressure to obtain a time course of a filtered respiratory muscle pressure, and
determining the patient component of the airway, flow based on the time course of the filtered respiratory muscle pressure.

19. A process in accordance with claim 13, wherein a determination of a high-pass characteristic is performed for the adapted performance of the high-pass filtering at least one of:
based on the time course of the respiratory muscle pressure; and
based on a kinetic equation for the breathing circuit of the patient.

20. A process according to claim 19, wherein a computer program with a program code performs at least some of the process steps when the program code is executed on a computer, on a processor or on a programmable hardware component.
